(19) United States
(12) Patent Application Publication
(10) Pub. No.: US 2003/0186905 A1
(43) Pub. Date: Oct. 2, 2003

(54) Cis-acting regulatory nucleic acid sequences in the parathyroid hormone3' -untranslated region

(76) Inventors: Justin Silver, Motza Illit (IL); Tally Nevah, Jerusalem (IL)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/169,519

(22) PCT Filed: Jan. 2, 2001
(86) PCT No.: PCT/IL01/00006

(30) Foreign Application Priority Data
Jan. 3, 2000 (IL) 133875

(51) Int. Cl. A61K 48/00; C07H 21/04

(52) U.S. Cl. 514/44; 536/23.2

(57) ABSTRACT

The invention relates to an isolated cis-acting regulatory nucleic acid sequence comprising the 3′-UTR of pararhyroid hormone (PTH) gene and allelic variations, mutations or functionally equivalent fragments thereof. When this sequence is operably linked to a heterologous or homologous coding sequence of interest, it is capable of directing specific regulation of stability of the mRNA encoded by the linked heterologous or homologous coding sequence. The regulation of the stability of the mRNA is responsive to changes in serum levels of any one of calcium and phosphate and is further mediated by the binding of at least one PT protein or derivatives thereof to said cis-acting sequence. The invention further relates to DNA constructs, host cells screening methods using the cis acting sequences of the invention and pharmaceutical compositions thereof.

### FIELD OF THE INVENTION

The invention relates to cis acting regulatory nucleic acid sequences comprising sequences related to a conserved sequence in the parathyroid hormone (PTH) mRNA 3′-untranslated region (UTR). These sequences are capable of binding to parathyroid cytosolic proteins and to regulate stability of the mRNA More particularly, the invention relates to these cis-acting sequences and various uses thereof.

### BACKGROUND OF THE INVENTION

Parathyroid hormone (PTH) acts together with the biologically active metabolite of vitamin D, 1α,25-dihydroxyvitamin D (1,25(OH)₂D₃) [Silver and Kronenberg Parathyroid Hormone—Molecular Biology and Regulation, in Bilezikian, Raisz and Rodan (ed.), Principles of Bone Biology, Academic Press, San Diego (1996)] to maintain serum calcium within a narrow physiological range. A 7-transmembranous calcium-sensing receptor (CaSR) on the parathyroid (PT) cell recognizes small changes in serum ionized calcium and regulates PTH secretion [Brown et al., Nature 366: 575-580 (1993)]. Low serum calcium increases PTH secretion, PTH mRNA levels [Naveh-Many and Silver, J Clin Invest 86:1313-1319 (1990)] and if prolonged, PT cell proliferation [Naveh-Many et al., J Clin Invest 96:1786-1793 (1995)]. PTH then acts to correct serum calcium by mobilizing calcium from bone and increasing renal reabsorption of calcium. Phosphate also regulates the PT, with low serum phosphate decreasing serum PTH, PTH mRNA levels and parathyroid cell proliferation [Almaden et al., J Bone Miner Res 11:970-976 (1996); Kilav et al., J Clin Invest 96:327-333 (1995); Nielsen et al., Nephrol Dial Transplant 11:1762-1768 (1996); Slatopolsky et al., J Clin Invest 97:25342540 (1996)]. The mechanisms whereby calcium and phosphate regulate the parathyroids is important to the management of patients with chronic renal failure who develop severe hyperparathyroidism with overactivity of the PT gland, bone pain and increased mortality [Block et al., Am J Kidney Dis 31:607-617 (1998); Hammerstad et al., Clin Neuropharmacol 17:429-434 (1994); Silver et al., Nephrol Dial Transplant (in press)]. There is therefore great interest in understanding the regulation of the parathyroid by calcium and phosphate.

Post-transcriptional regulation of RNA stability is determined by the susceptibility of an RNA to degradation by cellular ribonucleases. Increasing evidence demonstrate that mRNA decay is an actively regulated process that determines gene expression. This process involves transacting protein factors that interact with specific cis elements in a mRNA and under different physiologic conditions lead to rapid decay or stability. Defined elements in mRNAs bind specific RNA binding proteins and have been shown to mediate in addition to RNA stability, subcellular localization and RNA translation. The information encoded by such elements in the RNA can be packaged as primary sequences and secondary or tertiary structures or a combination of both. The primary sequence of some of these cis acting elements is highly conserved amongst species. Many of the cis elements reside in the untranslated domains of mRNAs, though in the c-fos and c-myc mRNAs there are destabilizing coding region elements that seem to function independently of other AUUUA repeats in their 3′-UTRs.

Cis elements that determine mRNA stability or instability have been determined in a number of mRNAs. A well defined example is the adenosine-uridine-rich element (ARE). Repeats of this AUUUA pentamer in the 3′-UTR of mRNAs of various cytokines targeted them for rapid decay by their interaction with cytoplasmic trans factors [Brewer, Mol Cell Biol 11:2460-2466 (1991); Loflin et al., Genes Dev 13:1884-1897 (1999)]. However, the same cis element determines stability of the β-globin mRNA as a result of the binding of a different trans complex [Chklheudze et al., Mol Cell Biol 19:4572-4581 (1999); Kiledjian et al., Mol Cell Biol 17:4870-4876 (1997)]. In the brains of Alzheimer's patients there are increased levels of β-amyloid protein and often the amyloid precursor protein (APP) mRNA as well [Rajagopalan and Malter, Prog Nucleic Acid Res Mol Biol 56:257-286 (1997)]. A 29-base element in the 3′-UTR has been defined that is bound by trans factors and determines the APP mRNA decay [Rajagopalan and Malter, ibid.]. The iron response element (IRE) is a well defined cis element. This element in the ferritin 5′-UTR controls translation of this mRNA and in the transferrin receptor mRNA it is present in multiple reiterations where it regulates mRNA stability [Klausner et al., Cell 72:19-28 (1993); Schlegl et al., RNA 3:1159-1172 (1997)]. Iron regulatory protein-1 (IRP-1) controls the expression of several mRNAs by binding to iron-responsive elements (IREs) in their untranslated regions. In iron-replete cells, a 4Fe-4S cluster converts IRP-1 to cytoplasmic aconitase. IRE binding activity is restored by cluster loss in response to iron starvation or hypoxic stress [Gehring et al., J Biol Chem 274:6219-6225 (1999)]. Vascular endothelial growth factor mRNA is also stabilized by hypoxia. A cis element in the VEGF mRNA 3′-UTR is bound by the RNA-binding protein HuR and this binding is increased by hypoxia [Levy et al., J Biol Chem 273:6417-6423 (1998)]. Cis elements are usually present in the 3′-UTR of a mRNA but may also be present in the coding region [Ross, J., Microbiol Rev 59:423-450 (1995)]. The inventors have now determined a cis element in the PTH mRNA 3′-UTR that determines PTH mRNA stability in response to, changes in serum calcium and phosphate. Determination of protein interactions with this element will lead to an understanding of how the degrading and stabilizing factors regulate PTH mRNA half-life.

PTH gene expression is markedly increased by hypocalcemia and decreased by hypophosphatemia and these effects in vivo are post-transcriptional [Kilav et al., ibid. Moallem et al., J Biol Chem 273:5253-5259 (1998)]. The PTH cDNA consists of three exons coding for the 5′-UTR (exon I), the prepro region of PTH (exon II), and the structural hormone together with the 3′-UTR (exon III) [Hendy et al., Proc Nat Acad Sci USA 78:7365-7369 (1981); Kemper, B., CRC Crit Rev Biochem 19:353-379 (1986)]. The rat 3′-UTR is 239 nucleotides long out of the 712 nucleotides of the full-length PTH RNA [Kemper, ibid.]. The 3′-UTR is 42% conserved between human and rat, while the coding region is 78% conserved at the nucleotide level [Kemper, ibid.].

The PTH mRNA (PTH cDNA Accession No. X05721) contains 704 nucleotides, in which the 3′ UTR is from nucleotide 465 to nucleotide 704. The inventors have previously shown that the 60 terminal nucleotides of the PTH mRNA 3′-UTR (nucleotides 644 to 704) were necessary for protein RNA interaction and for the regulation of PTH mRNA stability by cytosolic PT proteins of rats fed a low calcium or a low phosphate diet in an in vitro degradation assay [Moallem et al., ibid.]. These results showed that an intact terminal 60 nucleotides region is necessary for protein binding to the PTH mRNA 3′-UTR is both necessary and the responsiveness to changes in PT proteins induced by changes in dietary calcium and phosphate. Reduction of PT proteins binding region to a, shorter sequence will make drug development process easier to conduct, because the shorter the sequences studied, the easier will it be to use combinatorial chemistry for drug discovery. The present invention is indeed directed to other, shorter sequences inside the protein binding region of the PTH mRNA 3′-UTR.

### SUMMARY OF THE INVENTION

The present invention relates to an isolated cis-acting regulatory nucleic acid sequence. This isolated cis-acting regulatory nucleic acid sequence comprises the 3′-UTR of parathyroid hormone BOTH) gene and allelic variations, mutations or functionally equivalent fragments thereof. Wven this sequence is operably linked to a heterologous or homologous coding sequence of interest, it is capable of directing specific regulation of stability of the mRNA encoded by said heterologous or homologous coding sequence of interest.

In one preferred embodiment, the regulation of the stability of mRNA encoded by said heterologous or homologous coding sequence of interest by the cis acting sequence of the invention, is responsive to changes in serum levels of calcium or phosphate. The regulation of the mRNA stability is further mediated by the binding of at least one PT protein and derivatives thereof to said isolated cis-acting sequence of the invention.

In another specifically preferred embodiment, the isolated cis-acting sequence of the invention, comprises a functional fragment of the 3′-UTR of the parathyroid hormone (PTH). More specifically, this sequence comprises the nucleotide sequence from 644 to −704 bp downstream of the parathyroid hormone (PTH) coding sequence.

In yet another specifically preferred embodiment, the isolated cis-acting sequence of the invention is a 60-nucleotide sequence substantially as denoted by SEQ ID NO:8 and allelic variations, mutations or functionally equivalent fragments thereof. Preferably, this isolated cis-acting sequence is a 40-nucleotide sequence substantially as denoted by SEQ ED NO:9 and allelic variations, mutations or functionally equivalent fragments thereof. Most preferably, said sequence is a 26-nucleotide sequence substantially as denoted by any one of SEQ ID NOs:10 to 14 and allelic variations, mutations or functionally equivalent fragments thereof.

According to another embodiment, the isolated cis-acting sequence of the invention may further be operably linked to heterologous or homologous coding sequence and optionally to additional control, promoting and/or regulatory elements.

More specifically, these heterologous or homologous coding sequences may encode a protein selected from the group consisting of reporter proteins, enzymes, hormones, growth factors, cytokines, structural proteins and industrially applicable proteins, or a protein which is itself a therapeutic product.

In a preferred embodiment, the isolated cis-acting sequence of the invention is operably linked to a homologous coding sequence, which is the parathyroid hormone (PTH coding sequence, substantially as denoted by the GenBank Accession No. X05721. Alternatively, the heterologous sequence may encode a reporter protein selected from the group consisting of green fluorescent protein (GFP), luciferase, secreted alkaline phosphatase (SEAP), β-galactosidase (β-gal), β-glucoronidase and a secreted protein such as GH.

According to a further aspect, the invention relates to a DNA construct comprising:

a. an isolated cis-acting regulatory nucleic acid sequence comprising the 3′-UTR of parathyroid hormone (PTH) gene and allelic variations, mutations or functionally equivalent fragments thereof, which sequence, when operably linked to a heterologous or homologous coding sequence of interest, is capable of directing specific regulation of stability of the mRNA encoded by said heterologous or homologous coding sequence of interest;

b. an operably linked heterologous or homologous coding sequence; and

c. additional optional control, promoting and/or regulatory elements.

A preferred embodiment of this aspect of the invention is a DNA construct in which said cis-acting regulatory nucleic acid sequence comprises a functional fragment of the 3′-UTR of the parathyroid hormone (PTH) comprising the nucleotide sequence from 644 to −704 bp downstream of the parathyroid hormone (PTH) coding sequence.

In a specifically preferred embodiment, the DNA construct comprises the cis acting sequence according to the invention.

According to another preferred embodiment, the DNA construct of the invention comprises a heterologous or homologous coding sequence. These coding sequences encode a protein selected from the group consisting of reporter proteins, enzymes, hormones, growth factors, cytokines, structural proteins and industrially applicable proteins, or a protein which is itself a therapeutic product.

In a specific embodiment, the DNA construct the invention comprises a homologous coding sequence that is operably linked to the cis acting sequence of the invention. This homologous coding sequence may be the parathyroid hormone (PT) coding sequence, substantially as denoted by the GenBank Accession No. X05721. Alternatively, the heterologous coding sequence may encode a reporter protein selected from the group consisting of green fluorescent protein (GFP), luciferase, secreted alkaline phosphatase (SEAP) β-galactosidase (β-gal), β-glucoronidase and a secreted protein such as GH.

According to another embodiment, the invention relates to an expression vector. This expression vector comprises a cis-acting regulatory nucleic acid sequence, or a DNA construct according to the invention, and a suitable DNA carrier, capable of transfecting a host cell with said cis-acting regulatory nucleic acid sequence.

The expression vector of the invention may further comprise additional expression, control, promoting and/or regulatory elements operably linked thereto.

Another aspect of the present invention relates to a host cell transfected with a DNA construct or an expression vector of the invention.

Still further, the invention relates to a complex comprising the cis acting sequence of the invention, bound to at least one PT protein or to at least one PT-protein-mimetic agent as herein defined.

In yet a further aspect, the invention relates to an agent that selectively binds to an RNA oligonucleotide and/or to a DNA a cis acting sequence according to the invention.

The agents according to the invention are capable of enhancing the affinity of the cis acting sequence of the invention to at least one PT protein.

Still further, the invention relates to agents that are capable of modulating the affinity of a cis acting sequence according to the invention to at least one PT protein. Such agents are capable of increasing or decreasing the affinity of the cis acting sequence according to the invention to at least one PT protein.

Additionally, the invention relates to agents capable of affecting the stability of a complex according to the invention.

The invention also relates to a method of screening for substances that specifically bind to a cis acting sequence comprising a nucleotide sequence identical with the nucleotide sequence of the 3′-untranslated region (UTR) of the gene encoding parathyroid hormone (PTH), comprising the steps of (a) providing a sample containing a combinatorial library of candidate substances; (b) depositing said cis acting sequence on a suitable solid phase carrier; (c) incubating the said sample with the deposited cis acting sequence obtained in step (b); (d) washing off any non-bound sample material; (e) separating bound material from said solid phase carrier; and (f) identifying the material obtained in step (e).

The screening method of the invention may be used, for example, for screening for the agents which affect the stability of a complex in accordance with the invention.

The present invention further relates to a method of screening for substances that specifically bind to an isolated cis-acting regulatory nucleic acid sequence comprising the 3′-UTR of parathyroid hormone (PTH) gene and allelic variations, mutations or functionally equivalent fragments thereof. This binding affects the regulation of mRNA stability by said cis-acting sequence. This method comprises the steps of (a) providing a host cell transformed with any one of an expression vector and a DNA construct according to the invention; (b) introducing a combinatorial library of candidate substances to said host cell, under conditions which lead to the regulation of stability of the mRNA encoded by said heterologous or homologous coding sequence of interest; (c) detecting an end point indicative of regulation of stability of said mRNA, wherein said regulation is affected by binding of said candidate substances expressed by a certain clone of the combinatorial library, to said cis-acting sequence; and (d) isolating said combinatorial library clones expressing a substance that binds said cis-acting sequence and affects the regulation of mRNA stability by said isolated cis-acting sequence.

According to an alternative embodiment, the invention relates to a method of screening for a substance which affects regulation of mRNA stability by the isolated cis-acting regulatory nucleic acid sequence comprising the 3′-UTR of parathyroid hormone (PTH) gene and allelic variations, mutations or functionally equivalent fragments thereof. This method comprises the steps of: (a) providing a host cell transformed with any one of an expression vector or a DNA construct of the invention; (b) exposing said host cell to any test substance under conditions which lead to the regulation of stability of the mRNA encoded by said heterologous or homologous coding sequence of interest; and (c) detecting an end point indicative of regulation of stability of said mRNA, affected by said test substance.

Preferably, both above-mentioned methods of the present invention utilize the cis-acting sequence of the invention. This cis-acting sequence, when operably linked to a heterologous or homologous coding sequence of interest, is capable of directing specific regulation of stability of the mRNA encoded by said heterologous or homologous coding sequence of interest.

Preferably, the host cell of the invention is used in the screening procedures.

In both methods of the invention, an indicative end point is preferably the expression of said operably linked heterologous or homologous coding sequence, that may serve as a reporter protein.

A specific example is the protein encoded the parathyroid hormone (PTH) coding sequence, substantially as denoted by the GenBank Accession No. X05721. Alternatively, the cis acting sequence of the invention is operably linked to a heterologous coding sequence which encodes a reporter protein selected from the group consisting of green fluorescent protein (GFP), luciferase, secreted alkaline phosphatase (SEAP), β-galactosidase (β-gal), β-glucoronidase and a secreted protein such as GH. Any test substance that affects the mRNA stabilizing properties of the cis acting sequence of the invention will change the expression of the reporter gene. Expression of the gene encoding said reporter protein leads to a visually detectable signal that can be easily quantified.

Still further, the invention relates to pharmaceutical compositions for the prevention and/or treatment of a disorder associated with abnormal function of the parathyroid gland, comprising as active ingredient a therapeutically effective amount of at least one natural PT protein.

In another embodiment, the invention relates to pharmaceutical compositions for the prevention and/or treatment of a disorder associated with abnormal function of parathyroid, comprising as active ingredient a therapeutically effective amount of at least one agent according to the invention.

The pharmaceutical compositions of the invention may be used for the treatment and/or prevention of overproduction or underproduction of PTH.

Further, the pharmaceutical compositions of the invention may be used for the treatment of a disorder associated with abnormal metabolism of or calcium and/or phosphate, for the treatment and/or prevention of bone diseases, particularly osteoporosis and for the treatment of chronic renal failure (CRF).

The invention also relates to antisera containing antibodies directed against an agent according to the invention.

### BRIEF DESCRIPTION OF THE FIGURES

FIGS. 1A-1B Different PTH mRNA 3′-UTR transcripts used for binding assays revealed the minimal sequence for protein binding by REMSA

1A—The full-length PTH mRNA 3′-UTR (234 nucleotides) and smaller transcripts which were tested for binding by REMSA (RNA Electrophoretic Mobility Shift Assay). The minimal sequence for binding (26 nucleotides) is shown. The binding (bin) of parathyroid proteins by each transcript is indicated on the right.

1B—REMSA for the binding of parathyroid cytosolic proteins to the 234 nucleotides (full-length), 100 nucleotides and 26 nucleotides transcripts of the 3′-UTR. The first 2 lanes for each transcript shows the free probe without and with RNase T1. The third and fourth lane for each transcript show the protein complex formed in the presence of proteins without and after RNase T1. Abbreviations: bin (binding), prot (proteins), pr (prob), f-len (full-length), deg (degraded).

FIGS. 2A-2D Competition experiment for the binding of PT proteins to the 3′-UTR by REMSA—A 26 nucleotides transcript is sufficient to compete for the binding of PT cytosolic proteins with the full-length PTH mRNA 3′-UTR

PT proteins (prot) were incubated with the full-length transcript either without (lane 2) or with 50× or 100× of:

2A—3′-UTR without the terminal 60 nucleotides;

2B—25× or 50× of the 26 nucleotides binding element;

2C—25× or 50× of the 63 nucleotides including the binding element;

2D—50× or 100× of the calcium sensing receptor (CaSR) 3′-UTR transcript. There was competition with transcripts B and C, which include the binding element, but not by A and D. Abbreviations: prot (proteins), ex (excess), unlab (unlabled), comp (complex), deg (degraded free transcript).

FIGS. 3A-3C Antisense oligonucleotides corresponding to the protein binding element prevent binding of PT proteins to the PTH mRNA 3′-UTR

3A—The nucleotide sequence corresponding to the terminal 100 nucleotides of the 3′-UTR and the single stranded antisense oligonucleotides used for binding interference. The 26 nucleotide protein binding element is shown in bold.

3B—REMSA for the binding of PT proteins to the 3′-UTR without and with antisense oligonucleotides. All the samples were treated with RNase T1. Lane 1 shows the free probe in the absence of protein. Lane 2 shows the protein-RNA complexes formed in the presence of protein. For lanes 3-8, the RNA transcripts were preincubated at 80° C. with the different antisense oligonucleotides (1-6) depicted in FIG. 3A and then protein binding was analyzed by REMSA. Preincubation with the antisense oligonucleotides 3-5, which correspond to the protein-binding element or parts of it, prevented protein binding to the PTH mRNA 8′-UTR.

3C—UV cross-linking analysis for the binding of PT proteins to the 3′-UTR without and with antisense oligonucleotides. The assay was performed without (first lane) or after preincubation with antisense oligonucleotides 1-6, as for FIG. 3B either after unfolding at 80° C. (above) or w/o heating to 80° C. (below). Pre-incubation with the antisense oligonucleotides 3-5, which correspond to the protein-binding element or parts of it, prevented protein binding to the PTH mRNA 3′-UTR only if the RNA was denatured at 80° C. Abbreviations: prot (protein), bin (binding), ele (element), a-sen oligo (antisense oligonucleotide).

FIG. 4 Diagram of the PTH cDNA and the protein-RNA binding sequence in the 3′-UTR

The protein-binding sequence is present and highly conserved in the PTH mRNA 3′-UTR of pig, mouse, human and dog. Nucleotides that are not identical to the rat sequence are shown as bold. Abbreviations: Ra (rat), Pi (pig), Mo (mouse), Hu (human), Do (dog).

FIGS. 5A-5C The 63 nucleotides protein-binding region of the PTH mRNA 3′-UTR imparted responsiveness of growth hormone (GE) mRNA to PT proteins from rats fed low calcium or low phosphate diets in an in vitro degradation assay

5A—Schematic representation of the GH mRNA (above) and the chimeric GH mRNA containing the PTH 3′-UTR 63 nucleotide element inserted at the end of the GH coding region (below).

5B—In vitro degradation assay for labeled transcripts for PTH (top), GH (middle) and GH+63 nucleotide of the PTH 3′-UTR (bottom) with PT proteins from rats fed a normal low calcium or low phosphate diet. At timed intervals samples were removed for RNA analysis.

5C—Time response curves of transcripts for GH and GH+63 nucleotides of the PTH 3′-UTR after incubation with PT cytosolic proteins as in B. Each point represents the mean±SE of 3 different experiments. The PTH 63 nucleotides inserted into the GE mRNA resulted in stabilization of the transcript with low calcium PT proteins and destabilization with low phosphate PT proteins, similar to the native PTH transcript. The native GH transcript was not affected by the PT proteins from the different diets.

Abbreviations: T (time), norm (normal), l (low), ca (calcium), ph (phosphate), min (minutes), Trans-rem (transcript remaining).

FIG. 6A-B Insertion of the 63 nt protein-binding region of the PTH mRNA 3′-UTR into a random pCRII RNA

Insertion of the 63 nt protein-binding region of the PTH mRNA 3′-UTR into a random pCRII RNA resulted in decreased RNA stability that was dependent upon protein binding.

6A—cDNA fragments corresponding to 63 or 38 nt of the PTH mRNA 3′-UTR, were inserted into a polylinker of pCRII.

6B—Chimeric transcripts and a transcript of the polylinker were analyzed by in vitro degradation with PT proteins from normal rats. The last 3 lanes show the different transcripts at 60 min without added PT protein. The chimeric 63 nt transcript was less stable than the native and the chimeric 38 nt transcript. Abbreviations: T(time), trans (transcript), prot (protein)

FIGS. 7A-7B Purification of the PTH RNA 3′ UTR binding proteins by affinity chromatography

7A—Identification by TV cross-linking of eluates from a PTH RNA 3′-UTR affinity column. The proteins which bound the 3′-UTR were eluted with increasing salt concentrations and binding to the 3′-UTR was examined by U.V. cross-linking. The fractions that showed maximal binding were eluted at NaCl concentrations of 230-550 nM. The arrows indicate the three RNA-protein bands that are also present when parathyroid proteins are studied for binding. Molecular weight markers are indicated on the right.

7B—Northwestern blot of proteins from concentrated fractions elated from the affinity columns identified a 50 kDa protein which bound the PTT 3′-UTR. A sample of the proteins from the combined positive fractions was run on an SDS-polyacrylamide gel and then transferred to a nitrocellulose membrane. The membrane was first stained with Ponceau (left) to identify protein bands and then the membrane was incubated with radiolabeled PTH 3′-UTR for Northwestern analysis (right). There was prominent binding of labeled RNA to several bands including two proteins of ˜50 kDa. Abbreviations: elu (eluate), Pon (Ponceau), stain (staining).

FIG. 8 Stabilizing effect of eluate from the RNA column on the degradation in vitro of PTH mRNA by hypophosphatemic rat parathyroid proteins

The full-length radiolabeled PTH mRNA, transcript was incubated with cytosolic parathyroid protein extracts (10 μg) from hypophosphatemic rats without or with the addition of 200 and 400 ng protein of the eluate from the RNA column. The proteins used were eluted from an RNA column as in FIG. 7 at a 250 nM salt concentration. At timed intervals samples were extracted, run on agarose gels and autoradiographed to measure the intact transcript remaining. There was a dose dependent stabilization with added eluate. Abbreviations: elu (eluate), T (time).

FIG. 9 Recombinant p40^{AUF1} binds PTH 3′-UTR by REMSA

Increasing concentrations of recombinant p40^{AUF1} (AUF1) resulted in a shift of the PTH 3′-UTR transcript, which without protein ran as two bands. It is believed that the two bands represent secondary structure within some of the substrate molecules.

FIGS. 10A-B Stabilizing effect of p40^{AUF1} on the degradation in vitro of PTH mRNA by hypophosphatemic rat parathyroid proteins

The full-length radiolabeled PTH mRNA was incubated with cytosolic parathyroid protein extracts (10 μg) from hypophosphatemic rats and at timed intervals samples were extracted, run on agarose gels and autoradiographed to measure the intact transcript remaining.

10A—Degradation in the presence of increasing doses of recombinant p40^{AUF1}. p40^{AUF1} stabilized the PTH transcript dose-dependently. Addition of eluate (200 ng), prepared as in FIG. 3, together with 10 ng of p40^{AUF1} stabilized the PTH transcript at doses that alone had no effect.

10B—Degradation with PT proteins from normal and hypophosphatemic (-P) rats, without and with added recombinant P40^{AUF1} (200 ng), or BSA (6 μg), or LC8 (6 μg). Recombinant p40^{AUF1}, but not bovine serum albumin (BSA) or dynein light chain (LC8) stabilized the PTH transcript. Abbreviations: elu (eluate), T (time).

### DETAILED DESCRIPTION OF THE INVENTION

As mentioned in the Background of the Invention, the inventors have previously shown that cytosolic proteins from parathyroids bind to the 3′-UTR of rat PTH mRNA and regulate its stability [Moallem et al., ibid.]. In search for a factor which may help regulate PTH mRNA, the inventors have now identified by binding assays, competition experiments and oligonucleotides binding interference analysis, a novel 26-nucleotide cis element in the 3′-UTR of PTH mRNA (nucleotides 624 to 649), that binds parathyroid (PT) protein/s and affects serum levels of PTH.

Thus, in a first aspect the present invention relates to an isolated cis-acting regulatory nucleic acid sequence. This isolated cis-acting regulatory nucleic acid sequence comprises the 3′-UTR of parathyroid hormone (PTH) gene and allelic variations, mutations or functionally equivalent fragments thereof. When this sequence is operably inked to a heterologous or homologous coding sequence of interest, it is capable of directing specific regulation of stability of the mRNA encoded by said heterologous or homologous coding sequence of interest.

The term “isolated” as also used herein with respect to nucleic acids, such as DNA or RNA, refers to molecules separated from other DNAs, or RNAs, respectively, that are present in the natural source of the macromolecule.

As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides.

The terms derivatives and functional derivatives as used herein mean oligonucleotides with any insertions, deletions, substitutions and modifications that are capable of binding at least one parathyroid protein (PT-protein) (hereafter referred to as “derivative/s”).

In one preferred embodiment, the regulation of the stability of mRNA operably linked to the cis acting sequence of the invention by said sequence is responsive to changes in serum levels of calcium or phosphate. Thus, decrease in serum levels of calcium results in increase in the stability of the mRNA operably linked to said sequence, whereas increase in serum level of phosphate results in decrease in the stability of said mRNA. The regulation of the mRNA stability is further mediated by the binding of at least one PT protein and derivatives thereof to said isolated cis-acting sequence of the invention.

In another specifically preferred embodiment, the isolated cis-acting sequence of the invention, comprises a functional fragment of the 3′-UTR of the parathyroid hormone (PTH). More specifically, this sequence comprises the nucleotide sequence from 644 to −704 bp downstream of the parathyroid hormone (PTH) coding sequence.

In yet another specifically preferred embodiment, the isolated cis-acting sequence of the invention is a 63-nucleotide sequence substantially as denoted by any one of SEQ ID NOs:1 and 8 and allelic variations, mutations or functionally equivalent fragments thereof. Preferably, this isolated cis-acting sequence is a 40-nucleotide sequence substantially as denoted by any one of SEQ ID NOs:2 and 9 and allelic variations, mutations or functionally equivalent fragments thereof Most preferably, said sequence is a 26-nucleotide sequence substantially as denoted by any one of SEQ ID NOs:3 to 7 and 10 to 14 and allelic variations, mutations or functionally equivalent fragments thereof.

The cis acting nucleic acid sequence of the present invention may by an RNA oligonucleotide (as denoted by SEQ ID NOs:1 to 7), or a DNA oligonucleotide (as denoted by SEQ ID NOs:8 to 14).

A further example of a functional derivative of the 63-nucleotide element of the invention is the 26-nucleotide cis element. Sequence analysis of the PTH mRNA 3′-UTR of different species revealed a striking preservation of the cis element of the invention in rat, pig, mouse, human and dog. The homology amongst these species varies between from about 80 to about 100%, as compared to a much lower homology of ˜40% in the overall 3′-UTR sequences [Kemper, ibid.]. This finding suggests that this cis binding element may represent a functional unit that has been evolutionarily conserved.

Thus, sequence analysis (GAP program of GCG (Madison, Wis., USA)) of the 26-nucleotides cis binding element in the PTH mRNA revealed high conservation of the rat element in the rat PTH mRNA 3′-UTR to pig (26 of 26 nucleotides), mouse (23 of 26 nucleotides), human (19 of 26 nucleotides) and dog (19 of 26 nucleotides), with human and pig being identical (FIG. 4 and Table 1). The very high conservation of this sequence, that lies outside of the coding region, amongst different species, suggests a functional role for this element. The nucleotide sequences of these different species represent some functional derivatives in accordance with the present invention.

Therefore, in specific embodiments of the first aspect of the invention, the invention relates to a cis acting nucleic acid sequence substantially as denoted by SEQ ID NOs:3 and 8 (rat), SEQ ID NOs:4 and 11 (human), SEQ ID NOs:5 and 12 (mouse), SEQ ID NOs:6 and 13 (dog) or SEQ ID NOs:7 and 14 (pig).

Table 1 lists the preferred oligonucleotides of the invention.

Nucleic acids which have a sequence that differs from the nucleotide sequence shown in any one of the sequences denoted by SEQ ID NOs:1 to 14 due to degeneracy in the genetic code are also within the scope of the invention. Such nucleic acids encode functionally equivalent fragments (i.e., a fragment having a biological activity of mRNA stabilizing cis-acting element that is capable of directing regulation of stability of an operably linked mRNA thereto) but that differs in sequence from said sequence listings due to degeneracy in the genetic code, mutations or to polymorphism.

Therefore, the term “functional” as used herein is to be understood as any such sequence which would have a specific activity of mRNA stabilizing element.

Nucleic acid fragments within the scope of the present invention also include those capable of hybridizing under high or low stringency conditions with nucleic acids from other species for use in screening protocols to detect any additional elements capable of directing regulation of stability of an operably linked mRNA within the 3′-UTR of the PTH gene or its homologs, including alternate isoforms, e.g. mRNA splicing variants. Nucleic acids within the scope of the invention may also contain linker sequences, modified restriction endonuclease sites and other sequences useful for molecular cloning, expression or purification or recombinant forms of any heterologous or homologous coding sequences according to the invention.

According to another embodiment the isolated cis-acting sequence of the invention may further operably linked to heterologous or homologous coding sequence and optionally to additional control, promoting and/or regulatory elements.

Operably linked is intended to mean that the cis-acting nucleotide sequence is linked to a heterlogous or homologous coding sequence in a manner which permits expression of such coding sequence, when the appropriate molecules (e.g., PT proteins) are bound to the regulatory sequence(s) and regulate the mRNA stability.

As used herein, the term “homologous or heterologous coding sequence” or “gene” refers to a nucleic acid comprising an open reading frame encoding any desired gene, including both exon and (optionally) intron sequences.

The term “intron” refers to a DNA sequence present in a given gene, which is not translated into protein and is generally, found between exons.

More specifically, these heterologous or homologous coding sequences may encode a protein selected from the group consisting of reporter proteins, enzymes, hormones, growth factors, cytokines, structural proteins and industrially applicable proteins, and protein which are per se therapeutic products.

In a preferred embodiment, the isolated cis-acting sequence of the invention is operably linked to a homologous coding sequence, which is the parathyroid hormone (PTH) coding sequence, substantially as denoted by the GenBank Accession No. X05721.

Nucleic acids which have a sequence that differs from the nucleotide sequence shown in the sequence of GenBank Accession Number X05721, due to degeneracy in the genetic code are also within the scope of the invention. Such nucleic acids encode functionally equivalent peptides (i.e., a peptide having the biological activity of PTH), but differing in sequence from said sequence listings due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of PTH protein.

However, it is expected that DNA sequence polymorphisms that do lead to changes in the amino acid sequences of the subject PTH will exist among vertebrates. One skilled in the art will appreciate that these variations in one or more nucleotides (up to about 3-5% of the nucleotides) of the nucleic acids encoding PTH polypeptides PTH may exist among individuals of a given species due to natural allelic variation. Any and all such nucleotide variations and resulting amino acid polymorphisms are within the scope of this invention.

In another preferred embodiment, the isolated cis-acting sequence of the invention may be linked to a heterologous coding sequence encoding a reporter protein selected from the group consisting of green fluorescent protein (GFP), luciferase, secreted alkaline phosphatase (SEAP), β-galactosidase (β-gal), β-glucoronidase and a secreted protein such as GH.

According to a further aspect, the invention relates to a DNA construct comprising an isolated cis-acting regulatory nucleic acid sequence according to the invention; an operably linked heterologous or homologous coding sequence; and optionally additional control, promoting and/or regulatory elements.

Accordingly, the term control and regulatory elements includes promoters, terminators and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operatively linked to it may be used in these vectors to express DNA sequences encoding the PTH protein or any other desired protein of this invention.

Preferably, the invention relates to a DNA construct wherein said cis-acting regulatory nucleic acid sequence comprises a functional fragment of the 3′UTR of the parathyroid hormone (PTH) comprising the nucleotide sequence from 644 to −704 bp downstream of the parathyroid hormone (PTH) coding sequence.

In specifically preferred DNA constructs according to the invention the said functional fragment is a 60-nucleotide sequence substantially as denoted by SEQ ID NO:8 and allelic variations, mutations or functionally equivalent fragments thereof. Preferably, said functional fragment is a 40-nucleotide sequence substantially as denoted by SEQ ID NO:9 and allelic variations, mutations or functionally equivalent fragments thereof. Most preferably, said functional fragment is a 26-nucleotide sequence substantially as denoted by any one of SEQ ID NOs:10 to 14 and allelic variations, mutations or functionally equivalent fragments thereof.

According to another preferred embodiment, the DNA construct of the invention comprises heterologous or homologous coding sequence. These coding sequences encode a protein selected from the group consisting of reporter proteins, enzymes, hormones, growth factors, cytokines, structural proteins and industrially applicable proteins, or is itself a therapeutic product.

In a specific embodiment, the DNA construct the invention comprises as said homologous coding sequence the parathyroid hormone (:PTH) coding sequence, substantially as denoted by the GenBank Accession No. X05721.

It is to be appreciated that this DNA construct of the invention may be used for preparation of a pharmaceutical composition for treatment, particularly by gene therapy, of pathological conditions such as chronic renal failure.

For gene therapy purpose the DNA constructs of the invention may be naked (i.e., not encapsulated), provided as a formulation of DNA and cationic compounds (e.g., dextran sulfate), or may be contained within liposomes. Alternatively, the DNA constructs of the invention can be pneumatically delivered using a “gene gun” and associated techniques which are well known in the art [Fynan et al., Proc Natl Acad Sci USA 90:11478-11482, (1993)].

In an alternative embodiment, the DNA construct of the invention may comprise a heterologous coding sequence operably lied to the cis-acting element of the invention which encodes for example, a reporter protein selected from the group consisting of green fluorescent protein (GFP), luciferase, secreted alkaline phosphatase (SEAP), β-galactosidase (β-gal), β-glucoronidase and a secreted protein such as GH.

According to another aspect, the invention relates to an expression vector comprising a cis-acting regulatory nucleic acid sequence, or the DNA construct according to the invention, and a suitable DNA carrier, capable of transfecting a host cell with said cis-acting regulatory nucleic acid sequence.

“Expression vectors”, as used herein, encompass plasmids, viruses, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector containing cells. Plasmids are the most commonly used form of vector but other forms of vectors which serves an equivalent function and which are, or become, known in the art are suitable for use herein. See, e.g., Pouwels et al. Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988), which are incorporated herein by reference.

In general, such vectors contain in addition specific genes, which are capable of providing phenotypic selection in transformed cells. The use of eukaryotic viral expression vectors to express the genes coding for the polypeptides of the present invention are also contemplated.

Expression vectors of the invention may be administered in any biologically effective carrier, e.g. any formulation or composition capable of effectively delivering to the cells in vivo. Approaches include insertion of the subject DNA constructs of the invention in viral vectors including recombinant retroviruses, adenovirus, adeno-associated virus, and herpes simplex virus-1, or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct of CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that the particular expression vector provided for in vivo transduction of any homologous or heterologous coding sequence of the invention, are also useful for in vitro transduction cells.

The expression vector of the invention, further comprising additional expression, control, promoting and/or regulatory elements operably liked thereto.

Another aspect of the present invention relates to a host cell transfected with a DNA construct or the expression vector of the invention.

The terms “host cells” or “transfected host cells” are used interchangeably herein. It is understood that such terms refer not only to the particular subject cells but to the progeny or potential progeny of such a cell. Because certain modification may occur in succeeding generation due to either mutation or environmental influences, such progeny may not, in fact, be identical to the parent cell but are still included within the scope of the term as used herein.

As used herein, the term “transfection” means the introduction of a nucleic acid, e.g., the DNA construct or an expression vector, into a recipient cells by nucleic acid-mediated gene transfer. “Transformation”, as used herein, refers to a process in which a cell's genotype is changed as a result of the cellular uptake of exogenous DNA or RNA. Ligating a polynucleotide sequence into a gene construct, such as an expression vector, and transforming or transfecting host cells with the vector are standard procedures used are well-known in the art.

This invention also pertains to a host cell transfected with the expression vector or DNA construct of the present invention. The host cell suitable for expression of the DNA constructs and the expression vectors of the invention may be an eukaryotic cell, selected for example amongst-eukaryotic (east, avian, insect or mammalian) cells. More preferably, a suitable host cell would be a mammalian cell.

According to one particular embodiment, the mammalian host cell of the invention may be a PT (parathyroid) cell. Alternatively, the mammalian host cell of the invention may be selected from the group consisting of COS7, HEK (293T) and CHO cell lines.

It is to be appreciated that the transformed host cells genetically modified by the DNA construct or expression vectors of the invention may also be used for cell transplantation therapies. These cells are transplanted into a patient, e.g., to replace the destroyed or malfunctioning cells in the patient or to produce the desirable gene products. The genetically modified cells are preferably of the same species as the host into which they will be transplanted. Generally, mammalian target cells are used for treating mammalian subjects. Thus, in the case of a human patient, the cells are preferably human.

The target cells can be adult or precursor cells. Precursor cells are cells, which are capable of differentiating, e.g., into an entire organ or into a part of an organ, such cells, which are capable of generating or differentiating to form a particular tissue (e.g., muscle, skin, heart, brain, uterus, and blood).

As will be shown in the following Examples, and particularly Example 4, the novel 26-nucleotide cis element affects PTH production. Therefore, the cis acting nucleic acid sequence of the invention may be used as a tool for screening of combinatorial libraries in order to isolate potential drugs that would mimic PT proteins action or modulate PTH production.

One group of such drugs might be PT protein mimetic agents, i.e. substances capable of mimicking PT protein action, particularly, by binding to PTH mRNA and protecting it from degradation. A second group of potential drugs are agents that modulate PTH mRNA-PT protein association. Such drugs may have clinical uses in the treatment of disorders associated with abnormal parathyroid function and with abnormal serum calcium or phosphate levels.

Thus, in a further aspect the invention relates to functional analogues of PT proteins, also referred to as PT-protein mimetic agents. The terms analogues and functional analogues as used herein are to be taken to mean PT proteins with any insertions, deletions, substitutions and modifications that are capable of binding to a cis acting nucleic acid sequence of the invention and their functional derivatives. Upon binding, these agents protect the nucleic acids from nuclease degradation, thus mimicking PT-protein action.

Alternatively, PT-protein mimetic agents according to the invention may be low molecular weight substances, which are capable of binding to a cis acting nucleic acid sequence of the invention or to a functional derivative thereof and protect them from nuclease degradation. Such substances can be naturally-occurring or synthetic.

In an additional aspect, the invention relates to a complex in which a cis acting nucleic acid sequence of the invention, or a functional derivative thereof is complexed with at least one PT-protein or a PT-protein mimetic agent according to the invention. The complexes of the invention may be used in various assays and screening methods, in order to identify substances which may increase or decrease the affinity of a cis acting nucleic acid sequence of the invention to a PT-protein, or a PT-protein mimetic agent in accordance with the invention.

In yet a further aspect, the invention relates to low molecular weight agents capable of binding to a cis acting nucleic acid sequence of the invention or functional derivatives thereof. Such agent may enhance or reduce the affinity of any of the cis acting nucleic acid sequence of the invention to any PT-protein, PT-protein mimetic agent or functional analogue of PT-protein. These agents are also referred to herein as modulators or modulating agents. Examples of such agents are antisense oligonucleotides (Example 2) or peptide nucleic acids (PNA) [Nielsen, P. E., Curr Opin Struct Biol 9(3):353-357 (1999); Tyler et al., Proc Nat Acad Sci USA 96(12):7053-7058 (1999)].

Still further, the invention relates to a method of screening for substances that selectively bind to a cis acting nucleic acid sequence of the invention and to functional derivatives of such nucleic acid. In a first step, the screening method of the invention comprises mixing the cis acting nucleic acid sequence of the invention or its functional derivative with a sample containing a combinatorial library of candidate substances and incubating the mixture. After incubation, the non-bound sample material is washed off and the bound material is isolated and identified. The method of the invention, either the nucleic acid or the combinatorial library is to be deposited on solid phase before mixing with the other reactant [Stevens et al., J Immunol 137(6):1937-1944 (1986)]. Other screening techniques, known to the man skilled in the art, may be adopted in the screening method of the invention.

The present invention further relates to a method of screening for substances that specifically bind to an isolated cis-acting regulatory nucleic acid sequence comprising the 3′-UTR of parathyroid hormone (PTH) gene and allelic variations, mutations or functionally equivalent fragments thereof This binding affects the regulation of mRNA stability by the cis-acting sequence. This method comprises the steps of (a) providing a host cell transformed with any one of an expression vector and a DNA construct comprising (i) said isolated cis-acting sequence; (ii) heterologous or homologous coding sequence operably linked to the sequence in (i); and (ii) operably linked additional control, promoting and/or regulatory elements; (b) introducing a combinatorial library of candidate substances to said host cell under conditions which lead to the regulation of stability of the mRNA encoded by said heterologous or homologous coding sequence of interest; (c) detecting an end point indicative of regulation of stability of said mRNA, wherein said regulation is affected by binding of said candidate substances expressed by a certain clone of the combinatorial library, to said cis-acting sequence; and (d) isolating said combinatorial library clones expressing a substance that binds said cis-acting sequence and affects the regulation of mRNA stability by said isolated cis-acting sequence.

In the above method, any candidate substance expressed by a certain clone of the combinatorial library introduced to the host cell of the invention may bind to the cis-element of the invention. In case the candidate substances binds to a cis-acting element of the invention, and this binding or interaction affects the mRNA stabilizing properties of said cis-acting element, the clone expressing that candidate substances may be isolated. As an indication, any candidate substances that affect the mRNA stabilizing properties of said cis-acting element, will change the expression of the heterologous or homologous coding sequence operably linked to the cis-acting sequence of the invention. Thus, measuring the expression of said heterologous or homologous coding sequence enables identification of the desired substances.

According to alternative embodiment the invention relates to a method of screening for a substance which affects regulation of mRNA stability by the isolated cis-acting regulatory nucleic acid sequence comprising the 3′-UTR of parathyroid hormone (PTH) gene and allelic variations, mutations or functionally equivalent fragments thereof. This method comprises the steps of: (a) providing a host cell transformed with any one of an expression vector or a DNA construct comprising (i) said isolated cis-acting sequence; (ii) heterologous or homologous coding sequence operably linked to said sequence in (i); and (iii) operably linked additional control, promoting and/or regulatory elements; (b) exposing said host cell to any test substance under conditions which lead to the regulation of stability of the mRNA encoded by said heterologous or homologous coding sequence of interest; and (c) detecting an end point indicative of regulation of stability of said mRNA, affected by said test substance.

In a preferred embodiment, both the above methods of the present invention utilize the cis-acting sequence of the invention. This cis-acting sequence, when operably linked to a heterologous or homologous coding sequence of interest, is capable of directing specific regulation of stability of the mRNA encoded by said heterologous or homologous coding sequence of interest.

In another preferred embodiment, in both methods of the invention the host cell of the invention is used for the screening purpose.

In both methods of the invention, an indicative end point, according to a specifically preferred embodiment, is the expression of said operably linked heterologous or homologous coding sequence. Thus, if the test substance affects the mRNA stabilizing properties of the cis-acting sequence of the present invention, the expression of the coding sequence that is operably linked thereto will change due to changes in its mRNA stability. In case that the test substance interacts directly with said cis acting sequence, and forms a complex which elevates the stability of the operably linked reporter coding sequence, the expression of said reporter protein will increase. If the test substance reduces the mRNA stability, the expression of the reporter protein will decrease.

A specific example for “reporter” protein is the protein encoded the parathyroid hormone (PTH) coding sequence, substantially as denoted by the GenBank Accession No. X05721. When this homologous sequence is operably linked to the cis acting sequence of the invention, the indicative end point is the expression of the PTH.

In alternative embodiment, more conventional reporter proteins may be used to generate an indicative end point. In these examples the cis acting sequence of the invention is operably linked to an heterologous coding sequence which encodes a reporter protein selected from the group consisting of secreted proteins such as GH or any other secreted protein, or visually detected reporter proteins such as green fluorescent protein (GFP), luciferase, secreted alkaline phosphatase (SEAP), β-galactosidase (β-gal), β-glucoronidase and a secreted protein such as GH. Any test substance that affects the mRNA stabilizing properties of the cis acting sequence of the invention will change the expression of the reporter gene. Expression of the gene encoding said reporter protein leads to a visually detectable signal that can be easily quantified.

In a further embodiment, the invention relates to a kit for carrying out the screening method of the invention. A kit according to the invention would carry all the components necessary for detecting a compound that might bind or facilitate binding of endogenous parathyroid proteins to the defined PTH responsive element. The 26, 40 or 63 nt PTH cis acting nucleic acid sequence in accordance with the invention, or any functional derivatives thereof would be bound to a chip, filter, bead or any other solid or liquid support system. The tethered cis acting nucleic acid sequence would either be labeled with a fluorescent or other colored dye, or a radioactive label, or not labeled. The array of bound cis acting nucleic acid sequence would then be incubated with test samples. These may be either natural or synthesized chemical compounds, which may be labeled with different dyes as described above, or unlabeled. After an incubation for different time intervals, for example 5 to 60 minutes, to as long as a number of hours, at different temperatures such as 20° C. to 37° C., as well as higher and lower temperatures, the samples are washed. The cis acting nucleic acid sequence bound to a test chemical or compound would then be detected by colorimetric, fluorometric or by radioactivity assays.

In an additional aspect, the invention relates to pharmaceutical compositions for the treatment of a disorder associated with abnormal function of parathyroid gland, comprising as active ingredient a therapeutically effective amount of at least one natural PT-protein.

More particularly, the invention relates to pharmaceutical compositions for the treatment of a disorder associated with abnormal function of parathyroid gland, comprising as active ingredient a therapeutically effective amount of at least one PT-protein mimetic agent.

Alternatively, the invention relates to pharmaceutical compositions for the treatment of a disorder associated with abnormal metabolism or resistance to calcium, phosphate or vitamin D or its derivatives, comprising as active ingredient a therapeutically effective amount of at least one parathyroid modulating agent according to the invention.

The terms “abnormal metabolism or resistance to calcium, phosphate or vitamin D or its derivatives” can mean overproduction or underproduction of PTH, caused by abnormal metabolism of calcium or phosphate.

In a further embodiment, the terms “abnormal metabolism or resistance to calcium, phosphate or vitamin D or its derivatives” are related to a disorder leading to bone diseases, particularly osteoporosis. Alternatively, such term is related to a disorder caused by chronic renal failure (CRF).

The “pharmaceutically effective amount” for purposes herein is that determined by such considerations as are known in the art. The amount must be sufficient to prevent harmful effects of PT gland abnormal function.

The pharmaceutical compositions of the invention can be prepared in dosage unit forms and may be prepared by any of the methods well known in the art of pharmacy.

The composition of the present invention may be administered directly to the patient to be treated or it may be desirable to conjugate it to carriers prior to its administration. Therapeutic formulations may be administered in any conventional dosage formulation. Formulations typically comprise at least one active ingredient, as defined above, together with one or more acceptable carriers thereof.

Each carrier should be both pharmaceutically and physiologically acceptable in the sense of being compatible with the other ingredients and not injurious to the patient. Formulations include those suitable for oral, rectal, nasal or parenteral (including subcutaneous, intramuscular, intravenous and intradermal) administration. The formulations may conveniently be presented in unit dosage form and may be prepared by any methods well known in the art of pharmacy.

In addition, the pharmaceutical compositions of the invention may further comprise pharmaceutically acceptable additives such as pharmaceutically acceptable carriers, excipients or stabilizers, and optionally other therapeutic constituents. Naturally, the pharmaceutically acceptable carriers, excipients or stabilizers are non-toxic to recipients at the dosages and concentrations employed.

The magnitude of therapeutic dose of the composition of the invention will be of course vary with the group of patients (age, sex, etc.), the nature of the condition to be treated and with the route administration and will be determined by the attending physician.

In addition, the invention also relates to an antiserum containing antibodies directed against any PT-protein mimetic agent or against any modulator of PT-protein binding.

As shown in the Examples, insertion of the protein-binding element of the invention (SEQ ID NO:1), that include the 5′ and 3′ flanking nucleotides, into growth hormone (GH) mRNA, conferred responsiveness of this RNA to calcium and phosphate, demonstrating that it can function independently of surrounding PTH mRNA sequences.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental procedures

General Techniques in Molecular Biology

A number of methods of the art of molecular biology are not detailed herein, as they are well known to the person of skill in the art. Such methods include site-directed mutagenesis, PCR cloning, expression of cDNAs, analysis of recombinant proteins or peptides, transformation of bacterial and yeast cells, transfection of mammalian cells, and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3rd ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al. (eds), John Wiley & Sons. Inc., New York, N.Y.

Animals

Weanling male Sabra rats were fed a normal calcium (0.6%), normal phosphate (0.3%) diet; a low calcium (0.02%), normal phosphate (0.3%) diet; or a low phosphate (0.02%), normal calcium (0.6%) diet (Teklad, Ill.) for 3 weeks. At 3 weeks the thyroparathyroid tissue was removed under pentobarbital anesthesia and blood samples were taken for serum calcium and phosphate. The low calcium diet resulted in a serum calcium of 4.5±0.8 mg/dl (control=11.1±0.4 mg/dl). The low phosphate diet resulted in a serum phosphate of 4.1±0.5 mg/dl (control=9.9±0.7 mg/dl) and serum calcium of 12.±0.8 mg/dl (control=10.9±0.9 mg/dl).

RNA Electrophoretic Mobility Shift Assays (REMSA)

Labeled RNA transcripts (5000 cpm) spanning different regions of the PTH 3′-UTR RNA were incubated with thyroparathyroid extracts (10 μg), in a final volume of 20 μl containing 10 mM HEPES, 3 mM MgCl₂, 40 mM KCl, 5% Glycerol and 1 mM DTT (binding buffer) for 10 mim at 4° C. In some experiments RNase T1 (Sigma Chemicals, St. Louis Mo.) was added for further 10 min at room temp to a final concentration of 150 u/ml to digest unprotected RNA. For competition experiments unlabeled RNA was added as indicated. The samples were run for 3 hours on a native polyacrylamide gel (4% polyacrylamide:bisacrylamide (70:1) in a cold room. RNA-protein binding was visualized by autoradiography of the dried gels.

UV Cross-Linking Assay

UV cross-linking assay was performed using 10 μg of S100 thyroparathyroid extracts as previously described. The proteins were incubated with ³²P-labeled RNA transcripts of the 3′-UTR of the PTH cDNA. After UV cross-linking, the samples were digested by RNase A, fractionated by SDS-PAGE and autoradiographed as previously described [Moallem et al., ibid.].

Cytoplasmic Protein Purification

Cytoplasmic thyroparathyroid proteins (S100) were extracted by the method of Dignam et al. [Nucl Acid Res 11:1475-1489 (1983)]. Tissues were removed from the rats and immediately washed in cold phosphate buffered saline (PBS). The tissue suspended in 5 volumes of buffer A containing 10 mM HEPES, 1.5 mM MgCl₂, 10 mM KCl, 0.5 mM DTT and 0.5 mM PMSF (phenylmethylsulfonyl fluoride) and incubated on ice for 10 min. After centrifugation at 600 g for 10 min (4° C.) the supernatant was carefully decanted, mixed with 0.1 volumes of buffer B containing 0.3 M HEPES, 1.4 M KCl and 0.03 M MgCl₂ and centrifuged (4° C.) at 100,000 g×1 h. (Beckman Type TL-100). For RNA degradation assays the S100 fraction was prepared by homogenizing the tissue with a polytron in 2 volumes of 10 mM Tris/HCl pH 7.4, 0.5 mM DTT, 10 mM KCl, 1.5 mM MgCl₂. 0.1 Volume of the extraction buffer (1.5 mM KCl, 15 mM MgCl₂, 100 mM Tris HCl pH 7.4, 6 mM DTT, was added and the homogenate was centrifuged at 14,000 g for 2 min. to pellet the nuclei. The supernatant was centrifuged at 100,000 g×1 hr. at 4° C. Cytoplasmic extracts were immediately frozen at −80° C. in aliquots. Protein concentration was determined by O.D. densitometry (595 μm wavelength) using a Bradford reagent (Bio Rad).

RNA Transcripts and Probes

Labeled and unlabeled RNA was transcribed from linearized plasmids using an RNA production kit (Promega, Wis.) and the appropriate RNA polymerases. The specific activity of the RNA probe was 0.5-1.0×10⁶ cpm/ng.

For competition experiments unlabeled RNA was transcribed similarly in the presence of 1 mM each of the four nucleotides. The RNA unlabeled RNA was quantified by visualization on a 2% agarose gel.

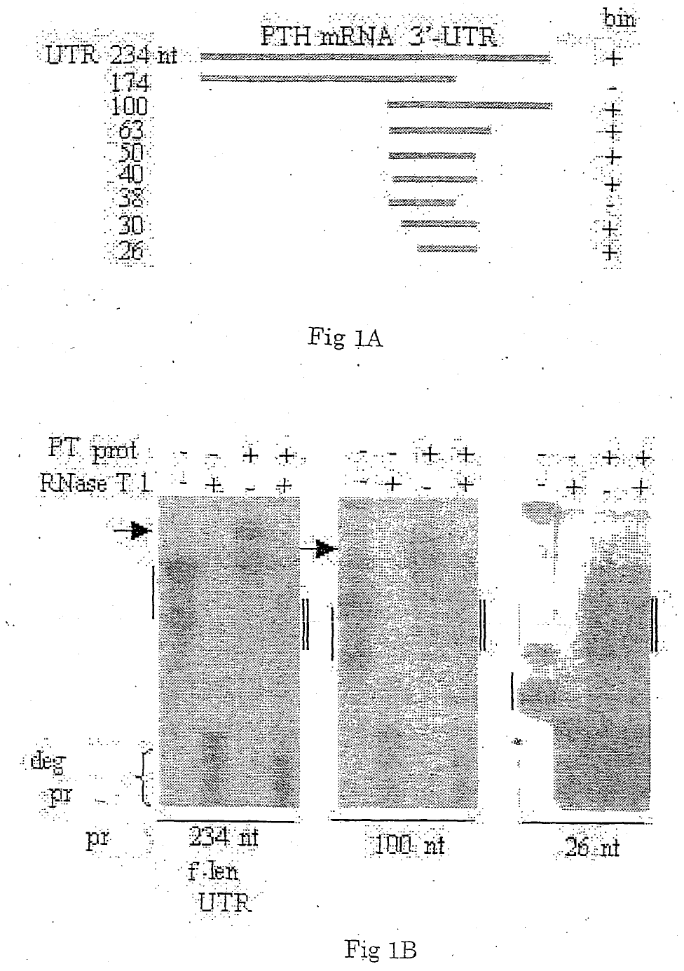

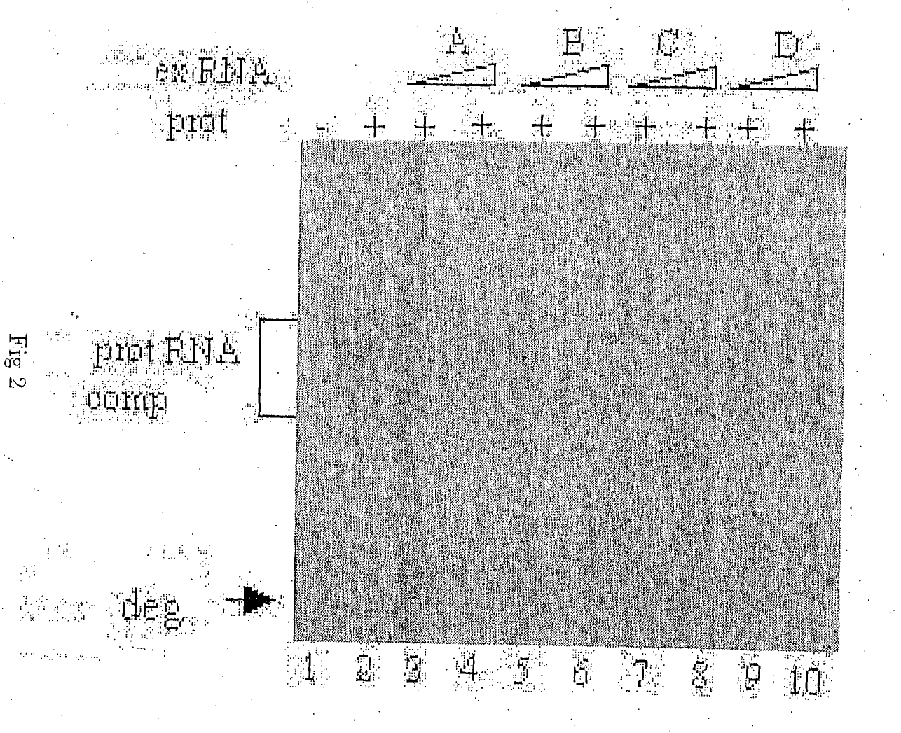

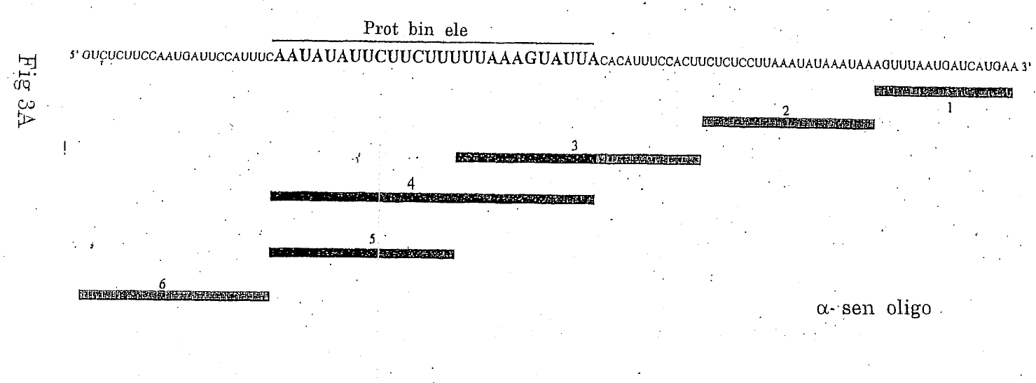

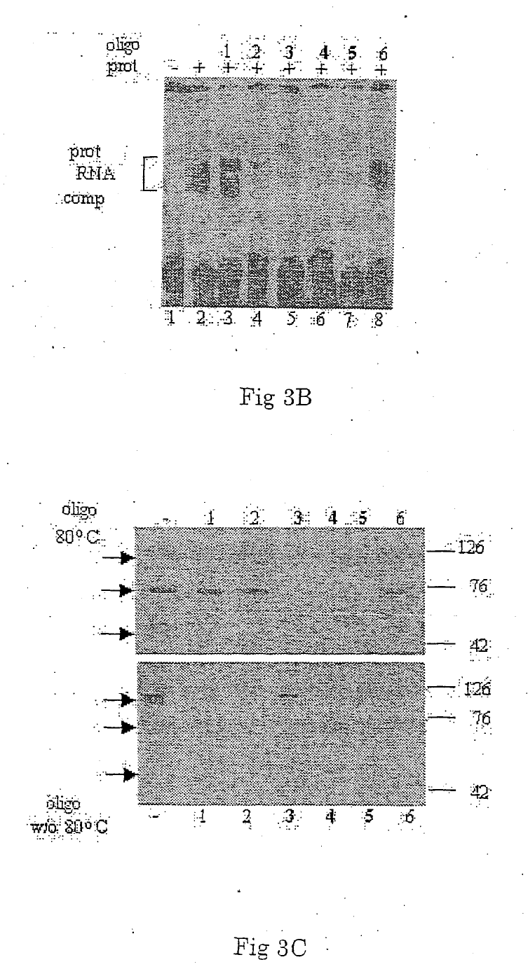

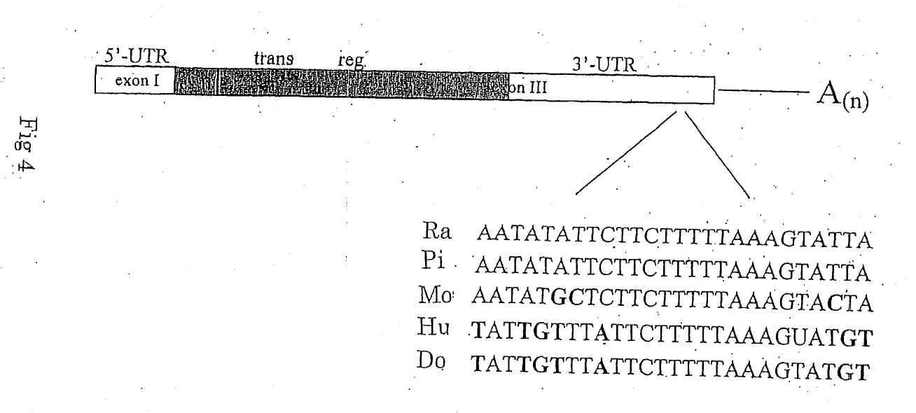

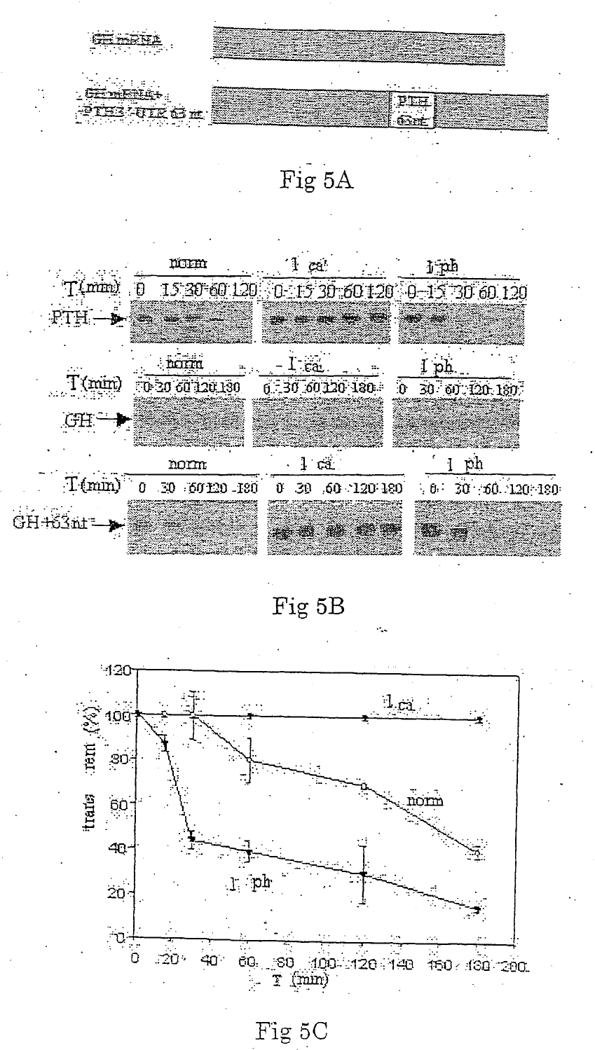

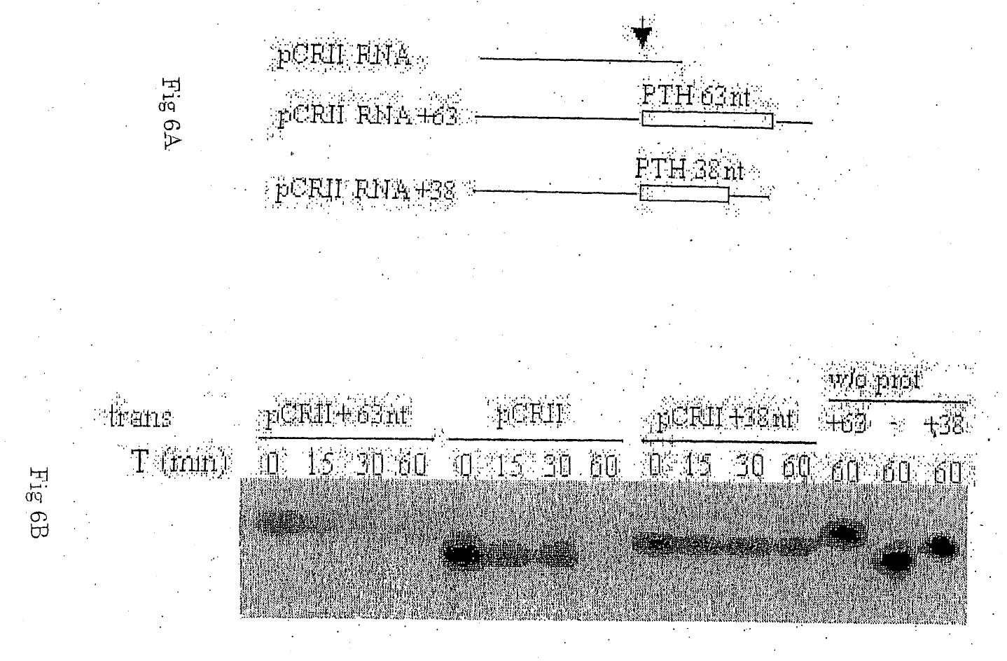

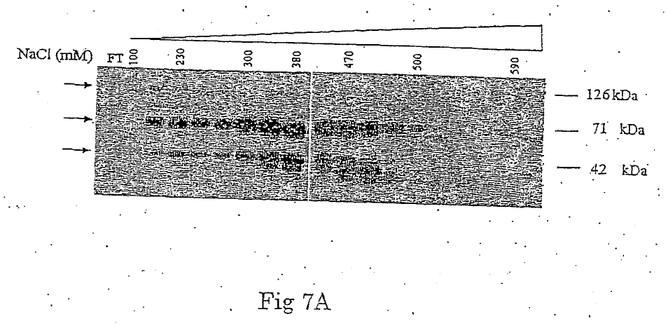

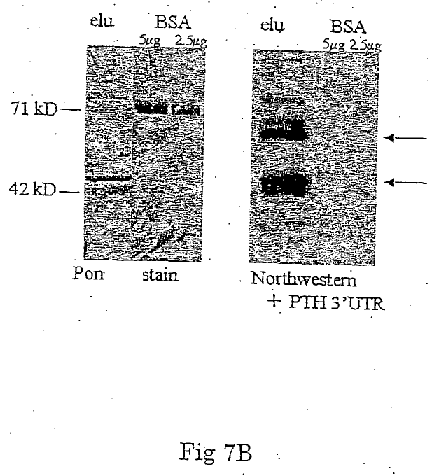

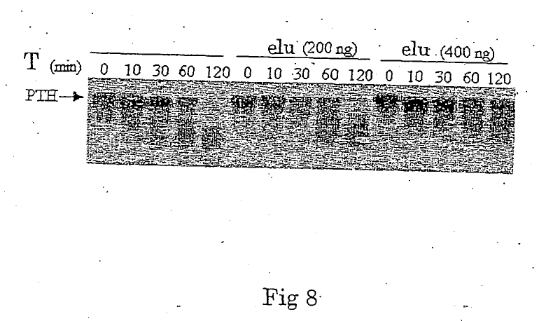

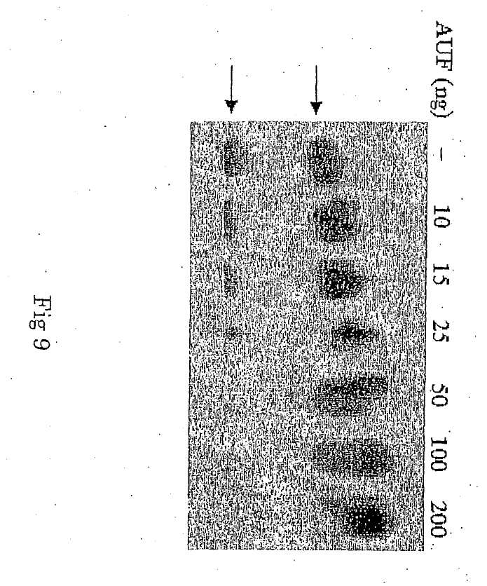

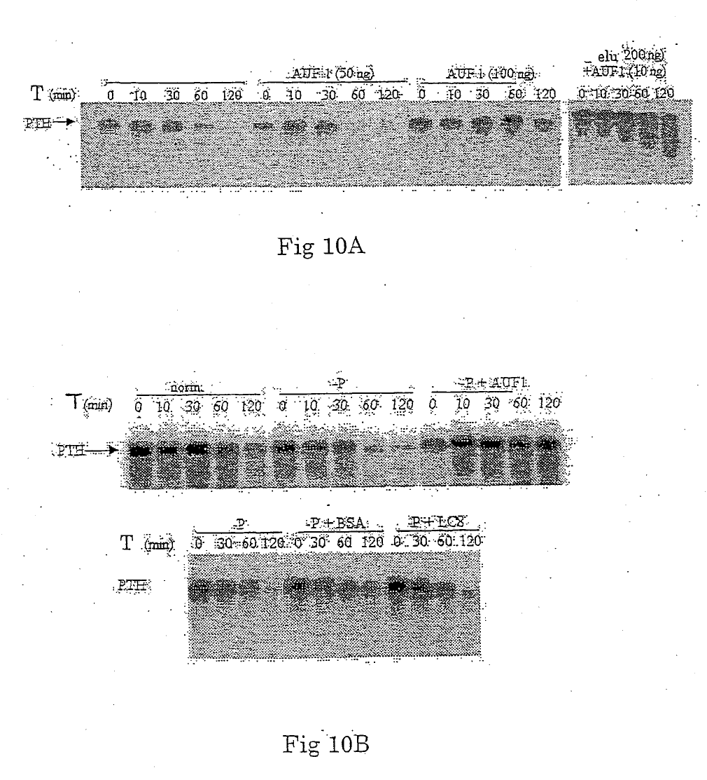

A linearized plasmid construct containing the full-length PTH cDNA in Bluescript KS (Invitrogen, Calif.) was used as previously described [Moallem et al., ibid.]. For the 3′-UTR of the PTH cDNA, a PCR product [Moallem et al., ibid.] was subcloned into PCRII (Invitrogen, Calif.). The transcripts of 100, 63, 50 and 40 nucleotide (FIG. 1A) were transcribed from PCR products using the oligonucleotides described in table 1 that were subcloned into PCRII (Invitrogen, Calif.). The transcript of 30 and 26 nucleotide (FIG. 1A) were prepared from annealed sense and antisense oligonucleotides that were constructed to include the T3 RNA polymerase sequence (underlined). For the 30 nucleotide the sense oligonucleotide was: ATTAACCCTCACTAAAGGGACATTTCAATATATTCTTCTTTTAAAGTAT T, and the antisense oligonucleotide was: AATACTTTAAAAAGAAGAATATATTGAAATG. For the 26 nucleotide the sense oligonucleotide was: ATTAACCCTCACTAAAGGGACAATATATTCTTCTTTTAAAGTATTA, and the antisense oligonucleotide was: TAATACTTAAAAAGAAGAATATATTG.

TABLE 2
Oligonucleotides used for PCR of parts of the PTH
cDNA (Numbers in parentheses designate SEQ ID NO)

| | 5′ oligo- | |
|---|---|---|
| | nucleotide | 3′ oligonucleotide |
| 100 nucleotide | GTCTCTTCCAATGAT | TTCATGATCATTAAACTT |
| transcript (15) | (16) | TA (17) |
| 63 nucleotide | GTCTCTTCCAATGAT | AAGTGGAAATGTGTAATA |
| transcript (8) | (16) | CTTTAA (18) |
| 50 nucleotide | GTCTCTTCCAATGAT | TAATACTTTAAAAAGAAG |
| transcript (19) | (16) | AATATATTG(20) |
| 40 nucleotide | AATGATTCCATTTCA | TAATACTTTAAAAAGAAG |
| transcript (9) | ATAT (21) | (22) |

The CaSR cDNA is 5.1 kb (4). For the CaSR 3′-UTR, the inventors subcloned the 3′-U=R into Bluescript II KS (Stratagene, La Jolla, Calif.) using a fragment obtained by restriction of the BoPCaRI cDNA in pSPORT (BRL, MD, USA) with NotI and SmaI.

Antisense Oligonucleotides and Binding Interference

For binding interference experiments antisense or sense oligonucleotides were mixed with the radiolabeled RNA, heated to 80° C. for 10 ml and cooled slowly to 25° C., before addition of protein extract and REMSA. In some experiments preincubation of the RNA and oligonucleotides was performed without heating of the RNA

Construction of the Chimeric GH mRNA Containing 63 Nucleotides of the PTH mRNA

3′-UTR. The 63 bp DNA corresponding to the PTH mRNA 3′-UTR 63 nucleotide transcript (FIG. 1A) that was subcloned into PCRII was excised and inserted into the SmaI site of the GH structural gene (FIG. 6A).

In Vitro R7A Degradation Assay

Preparation of S100 parathyroid protein extracts for the RNA degradation assay and the assay itself were performed as before [Moallem et al., ibid.]. 0.2×10⁶ cpm transcripts of PTH, GH or the chimeric GH/PTH 63 nucleotides RNAs were incubated with 10 μg of cytoplasmic extracts and 80 u/ml RNasin (Promega, Wis.) and at timed intervals samples were removed and extracted by TRI reagent (Molecular Research Center, OH). The labeled RNA from each sample was run on formaldehyde-agarose gels, transferred to Hybond membranes (Amersham, UK) and autoradiographed. The remaining undegraded transcripts at the different time points were quantified by densitometry.

Isolation and Identification of the 50 kDa Protein

S100 extracts were prepared from rat brain tissue. The tissue was removed from the rat under pentobarbital anesthesia and immediately washed in PBS buffer at 4° C. The tissue was homogenized with a polytron in one volume of S100 buffer (50 mM Tris pH 7.5, 25% glycerol, 100 mM KCl, 0.1 mM EDTA, 0.5 mM dithiothreitol, 0.5 mM phenylmethanylsulfonylfluoride). The homogenate was centrifuged at 12,000 g for 15 min at 4° C. and the supernatant was centrifuged again at 100,000 g for 1 h (Beckman type TL-100) at 4° C. The high speed supernatant (S100) was stored at −80° C. until it was used for protein purification and binding assays.

Heparin-sepharose (6 g) (Pharmacia, Piscataway, N.J) was used to prepare a 25 ml bed volume column. The heparin-sepharose column was washed with 250 ml of buffer B (50 mM Tris pH 7.8, 2 mM EDTA, 5% glycerol, 7 mM β-mercaptoethanol) containing 0.1 M NaCl. S100 brain tissue extract from 20 rats (300 mg) was applied to the column (x2). The column was washed with 550 nml of buffer B containing NaCl (0.1 M), and the bound proteins were eluted from the column by a step gradient of buffer B containing increasing NaCl concentrations (0.1-1 M).

The fractions were assayed for binding to the PTH 3′-UTR by UV cross-lining. Fractions that showed maximal binding to the PTH 3′-UTR eluted at 230-550 mM NaCl and were pooled. The pooled fractions were then loaded on a CNBr-activated Sepharose column bound to 200 μg of PTH mRNA 3′UTR that had been synthesized in vitro. The column was washed with Buffer B containing 0.1 M NaCl and the fractions were eluted with increasing NaCl concentrations (0.1-1 M) and assayed by a UV cross-linking assay. Fractions that showed maximal binding were pooled and concentrated using a Centricon 30 filter (Amicon, Beverley, Mass.). A sample was used to identify the RNA-binding proteins by Northwestern analysis with PTH 3′-UTR as a labeled probe. The pooled fractions were run on a preparative polyacrylamide gradient gel (7-12%) and stained with Coomassie blue. A 50 kDa band was excised from the gel, degraded with the endoprotease LysC, and the peptide products were analyzed by HPLC. Five peptides were microsequenced by Edman degradation.

Protein Gel Electrophoresis and Northwestern Analysis

Protein extracts were electrophoresed on sodium dodecyl sulfate (SDS) polyacrylamide gels [Laemmli, U. K., Nature 227:680-685 (1970)] and electrotransferred onto nitrocellulose membranes (0.2 μm, Schleicher & Schuell, Keene, N.M.).

For Northwestern assays, the membranes were pre-soaked in TBST (10 mM Tris pH 8, 150 mM NaCl, 0.05% Tween) and then incubated in a buffer containing 10 mM HEPES, pH 7.6, 40 mM KCl, 5% Glycerol, 1 mM DDT, 0.3 mM phenylmethylsulfonyl fluoride, 0.2% NP40, 0.5 M NaCl, 3 mM MgCl₂, 0.1 mM EDTA and 5 mg/ml BSA for 15 min at room temp. The membranes were washed twice in TNE buffer (10 mM Tris pH 7.5, 50 mM NaCl, 1 mM EDTA, 1 mM DTT) and then incubation was performed in binding buffer (10 mM HEPES, pH 7.6, 150 mM KCl, 5 mM MgCl₂, 0.2 mM DTI, 8% glycerol) supplemented with 100 μg/ml RNase free tRNA (Boehringer Mannheim, Germany) and the RNA probe (1×10⁶ cpm/ml) for 20 min. at 37° C. and then for 2 h at room temp. The membranes were washed twice at room temp for 5 mm with TNE buffer and RNA binding to the proteins was visualized by autoradiography.

Preparation of recombinant p40^{AUF1}

Recombinant p40^{AUF1} was prepared according to Wilson and Brewer [Wilson, G. M. and Brewer, G., Methods: A Companion to Methods in Enzymology 17:7483 (1999)] with some modifications. An E. coli DH5α clone containing pTrcHisB/p40^{AUF1} was induced to express plasmid encoded protein by culturing with 1 mM isopropyl-β-D-thiogalactopyranozide (IPTG) (MBI, Fermentas, N.Y.). His₆-AUF1 fusion polypeptide was purified by resuspending the bacterial pellet with HNTA buffer (1 M NaCl, 50 mM NaPO₄ buffer, pH 7.8, 1% Triton X-100, 10 μg/ml pepstatin A, 10 μg/ml leupeptin and 0.1 mM PMSF), sonication and centrifugation at 10000×rpm for 20 min at 4° C. The supernatant was added to 2 ml ProBond Resin (Invitrogen, Calif.) that had been prewashed twice with double distilled water and once with NTA buffer (300 mM NaCl, 50 mM NaPO₄ buffer pH 7.8, 1% Triton X-100, 10μ/ml pepstatin A, 10 μg/ml leupeptin, 0.1 mM PMSF) and rotated at 4° C. for 1 h. The beads were spun down, washed with NTA buffer and the His₆-AUF1 protein was eluted with increasing concentration of imidazole (25-300 AM) in NTA buffer. Lysozyme (Sigma, Mo.) was added at a final concentration of 100 μg/ml to the eluates containing the His₆AUF1 as the main polypeptide, and dialyzed against 100 vol of 10 mM Tris HCl pH 7.5, 4° C. for 5 h. The eluates were then concentrated by Centricon 30 (Amicon, Mass.) and the concentration of purified r AUF1 was determined by comparison with known amounts of BSA on Coomassie-stained SDS-PAGE gels.

### Example 1

### The Nature of 3′-UTR of PTH mRNA Interaction with PT Proteins

The nature of 3′-UTR of PTH mRNA interaction with PT proteins was studied in vitro by using two different binding assays. The purpose of these studies was to identify the minimal region required for PT proteins binding.

(1) RNA Electrophoretic Mobility Shift Assays (REMSA)

Parathyroid cytosolic proteins specifically bind the full-length PTH mRNA transcript and a transcript for the PTH mRNA 3′-UTR. A transcript that did not include the 60 terminal nucleotides did not bind proteins [Moallem et al., ibid.]. To identify the protein binding sequence in the PTH mRNA 3′-UTR the inventors analyzed the binding of parathyroid proteins to smaller RNA transcripts of the terminal 3′ of the PTH mRNA 3′-UTR (FIG. 1). Uniformly labeled transcripts were incubated with 10 mg PT cytosolic extract and the mixture resolved on native polyacrylamide gels for RNA electrophoretic mobility shift assays (REMSA). A transcript of 234 nucleotides consisting of the full-length UTR and transcripts of 100 (FIG. 1B), 63, 50 (not shown) and 40 nucleotides (not shown) of the distal 3′-UTR showed a large protein-RNA complex on REMSA with PT proteins (FIG. 1). This complex was reduced to a smaller complex after RNase T1 digestion of the bound protein RNA samples (FIG. 1B). Similar results were obtained when a transcript for the full-length PTH mRNA was analyzed (not shown). The smaller protein-RNA complex after RNase T1 was also formed when transcripts of 30 (not shown) and 26 nucleotides (FIG. 1B) were analyzed for binding to PT cytosolic proteins. However the binding of the 30 or 26 nucleotides transcript was the same with or without treatment with RNase T1 (FIG. 1B) and the large RNA-protein complex was not formed. These results show that a transcript of 40 nucleotides was necessary for the formation of the large protein RNA complex that was obtained when the full-length PTH mRNA 3′-UTR transcript was analyzed. A 26 nucleotide element was sufficient for protein binding and formed a complex that was similar to the complex formed with larger transcripts after treatment with RNase T1. Therefore, additional nucleotides in the 5′ of this element were necessary for formation of the large complex that was formed in the absence of RNase T1. Further experiments were performed to conform the minimal binding element.

To demonstrate the specificity of the binding of PT proteins to the protein-binding element the inventors used competitor RNAs from overlapping regions of the 3′-UTR. FIG. 2 shows a representative REMSA that demonstrates the binding of PT cytosolic proteins to the PTH mRNA 3′-UTR. Addition of excess unlabeled RNA transcript for the 3′-UTR that did not include the terminal 60 nucleotide of the 3′-UTR did not compete for binding of PT proteins to the PTH 3′-UTR. However, excess transcript of the 26 nucleotides element or of a 63 nucleotides that included the 26 nucleotides binding element both competed for protein binding to the 3′UTR. Excess of an unrelated transcript for the 3′-UTR of the CaSR mRNA, which is also expressed in the PT, did not compete for binding. These results indicate that the 26 nucleotides transcript was sufficient to compete for the binding of PT proteins to the PTH mRNA 3′-UTR.

The gel shift binding experiments identified the minimal binding sequence as a 26 nucleotides element in the PTH mRNA 3′-UTR. Gel shift experiments with PT proteins and the full-length PTH mRNA as well as the 234 nucleotides 3′-UTR, showed a large protein-RNA complex which was reduced to smaller complexes after RNase T1. These smaller complexes were similar to the binding of the 26 nucleotide element with or without RNase T1. The large complex was also obtained with smaller transcripts of 100 nucleotides down to 40 nucleotides that contained the binding element. These results indicate that the 26 nucleotides element is sufficient for protein binding and that the formation of the larger complex is dependent upon additional flanking sequences that are 5′ to the element. These sequences may be necessary to stabilize the protein-RNA interaction. The minimal element that shows protein binding is therefore the 26 nucleotides transcript. However, in the context of the full-length mRNA, a larger sequence of 40 nucleotides may represent the physiological sequence for protein-RNA interaction.

(2) UV Cross-Linking Assay

PT proteins from hypocalcemic rats show increased binding to the PTH mRNA 3′-UTR by mobility shift and UV cross-linking assays and this protein-RNA binding is decreased with hypophosphatemic PT proteins. Thus the level of protein-RNA binding directly correlates with PTH mRNA levels. Since there is no PT cell line, an in vitro PTH RNA stability assay was utilized. This assay showed stabilization of the transcript by hypocalcemic PT proteins and marked instability with PT hypophosphatemic proteins [Moallem et al., ibid.]. A PTH transcript that did not include the 3′-UTR was not degraded by parathyroid proteins in this assay. These studies show that there are instability regions in the PTH mRNA 3′-UTR that are protected by RNA binding proteins. This protein-RNA interaction determines PTH mRNA stability.

### Example 2

### Antisense Oligonucleotides and Binding Interference

To further characterize the protein binding element of 26 nucleotides in the PTH mRNA 3′UTR by competition experiments, the inventors designed short single stranded antisense DNA oligonucleotides complimentary to portions of the 3′-UTR and analyzed their effect on protein RNA binding by REMSA and UV cross-linking analysis.

FIG. 3A shows the sequence of the 100 terminal nucleotides of the PTH mRNA 3′-UTR including the 26 nucleotides of the proposed protein binding element. Antisense oligonucleotide sequences are shown in FIG. 3A. Corresponding sense oligonucleotides were also synthesized. A representative REMSA for the binding of cytosolic PT proteins to a transcript that had been pre-incubated with different anti-sense oligonucleotides is shown in FIG. 3B. The anti-sense oligonucleotides were annealed to the labeled 3′-UTR transcript that had been heated to 80° C. to unfold secondary structures in the RNA. PT protein extracts were then added and protein binding analyzed. Corresponding sense oligonucleotides were used as controls. FIG. 3B shows the 3′-UTR transcript without and with RNase T1 treatment and the protein-RNA complexes formed after addition of PT cytosolic extracts. Pre-incubation of the PTH mRNA 3′-UTR transcript with oligonucleotides 3, 4 and 5 (antisense oligonucleotides corresponding to the protein binding element) prevent binding of PT proteins to the PTH mRNA 3′-UTR. Oligonucleotides 1, 2 and 6 that did not span the protein binding sequences had no effect on protein binding. Pre-incubation with oligonucleotides spanning the 26 nucleotides element or part of this sequence with or without 3′ flanking sequences (oligonucleotides 3, 4 and 5) abolished the binding of PT proteins to the 3′-UTR. Corresponding sense oligonucleotides as well as double stranded DNA had no effect on protein-RNA complex formation (not shown). The effect of antisense oligonucleotides on protein binding to the 3′UTR was also analyzed by W cross-linking experiments. In this assay RNA-binding proteins from cytosolic extracts are cross-linked to labeled transcript in solution and complexes resolved by denaturing SDS PAGE. FIG. 3C shows that 3 cross-linked protein RNA complexes of ˜110, 60, 50 kDa were formed when a transcript for the PTH mRNA 3′-UTR was analyzed with PT protein extracts, as in previous reports [Moallem et al., ibid.]. When the transcript was denatured at 80° C. and pre-incubated with the antisense oligonucleotides the same inhibitory effect of the oligonucleotides corresponding to the binding region on protein binding was observed (FIG. 3C), similar to the REMSA (FIG. 3B). When the same TV cross-linking experiment was performed without denaturing the RNA by heating to 80° C. there was no effect of pre-incubation of the RNA with any of the oligonucleotides (FIG. 3C). The transcript of 26 nucleotides was sufficient to compete for the binding to the full-length PTH mRNA 3′-UTR. Single-strand antisense oligonucleotides sparing the binding element, or parts of it, interfered with the binding of PT proteins to the 3′-UTR. Together, these results indicate that the element of 26 nucleotides of the 3′-UTR contains the protein binding recognition sites and that this region plays a role in facilitating protein binding. Moreover, the interference of binding only after unfolding the RNA at 80° C. suggests that protein binding to this region is dependent on secondary structures in the RNA

### Example 3

### In Vitro RNA Degradation Assay

Since there is no appropriate PT cell line, the inventors performed in vitro degradation assays to measure the effect of PT cytosolic proteins on these transcripts. In this assay labeled transcripts were incubated with PT cytosolic proteins and at timed intervals samples taken and RNA extracted and run on gels to determine the amount of intact transcript remaining.

In the presence of protein there is gradual degradation of the RNA that is the net result of protective and degrading factors which are present in the protein extract. The degradation assay was first performed with the full-length PTH transcript and PT proteins from rats fed control low Ca or low P diets (FIG. 5B, top panel). With control PT proteins there was gradual degradation of the PTH transcript. With PT proteins from low, Ca rats the transcript was much more stable. There was rapid degradation with low P proteins (FIG. 5B). These results are similar to inventors previous results [Moallem et al., ibid.] and show that the in vitro degradation of PTH transcript correlates with PTH mRNA levels in vivo.

### Example 4

### Functional Analysis of the 26 Nucleotides Element from PTH mRNA 3′-UTR

To determine the functionality of the protein binding element in the PTH mRNA 3′-UTR, the inventors studied whether this element has a role in determining PTH mRNA levels and in particular if it is involved in the regulation of PTH mRNA stability by dietary calcium and phosphate deficiency.

The inventors have previously shown that dietary calcium deficiency resulted in a post-transcriptional 10-fold increase in PTH mRNA levels and dietary phosphate deficiency in a post-transcriptional 6-fold decrease in PTH mRNA levels. The inventors have shown that proteins that bind to the PTH mRNA 3′ UTR regulate these effects [Moallem et al., ibid.]. The inventors have now identified the protein binding sequence in the 3′-UTR as a 26 nucleotides element with its flanking sequences. To demonstrate that the protein binding region has a role in determining mRNA stability and response to Ca and P, the inventors inserted a fragment of 63 nucleotides (SEQ ID NO:1 and 8, (RNA and DNA, respectively)) of the PTH mRNA 3′-UTR, which contains the 26 nucleotides element, into the structural gene of human growth hormone (GH) (FIG. 5A). the inventors then analyzed the effect of this insertion on GH mRNA stability (FIGS. 5B and C).

To demonstrate the sufficiency of this region to confer PTH-like responsiveness to calcium and phosphate on another gene, a chimeric gene was constructed, The inventors inserted a 63 nucleotides fragment (SEQ ID NO:8), containing the 26 nucleotides binding region, into the structural gene of human growth hormone. Since there is no appropriate PT cell line the inventors performed in vitro degradation assays to measure the effect of PT cytosolic proteins on the half-life of GH and chimeric GH/PTH 63 nucleotides RNAs. The GH transcript was stable in the presence of PT proteins and its degradation was the same with PT proteins of rats fed a normal, low calcium or low phosphate diet. In contrast, the chimeric GH rRNA transcript containing the 63 nucleotides of the PTH mRNA responded to PT proteins from low Ca and P similar to the PTH mRNA The insertion of the 63 nucleotides of the PTH 3′-UTR also resulted in decreased stability of the GH transcript in the presence of PT proteins compared to the stability of GH mRNA, suggesting that it acts as an instability sequence.

When a transcript for the GH mRNA was analyzed with PT proteins from the different diets, there was no effect on GH degradation (FIG. 5B, middle panel). In addition, the GH transcript was more stable than the PTH transcript in the presence of PT proteins. The inventors then analyzed the chimeric GE transcript that was constructed to include the 63 nucleotides of PTH mRNA 3′-UTR. This transcript was now gradually degraded by PT proteins from control rats, more stable with PT proteins from low Ca rats and more rapidly degraded with PT proteins of low P rats (FIG. 5B, bottom panel and FIG. 5C), similar to the full-length PTH transcript. Inserting the 63 nucleotides of the PTH mRNA to GE RNA transcript resulted in the chimeric transcript responding to PT proteins from low Ca and P similar to the PTH mRNA. The insertion of the 63 nucleotides of the PTH 3′-UTR also resulted in decreased stability of the growth hormone transcript in the presence of PT proteins compared to the stability of GH mRNA, suggesting that it is an instability element (FIG. 5B). In addition, the protein binding sequences in the PTH mRNA 3′UTR were sufficient to confer responsiveness to changes in PT proteins induced by dietary Ca and P on mRNA of another gene.

To determine the specificity of the effect of the protein-binding region, the protein-binding segment of 63 nt was inserted into a random sequence, the pCRII polylinker. In addition a shorter PTH mRNA 3′-UTR RNA of 38 nt, that itself did not bind PT proteins (FIG. 1A) was also inserted at the same site into the pCRII polylinker (FIG. 6A). The stability of the polylinker and chimeric RNAs was determined in the in vitro degradation assay with PT proteins. The PTH mRNA 63 nt was recognized and cleaved more rapidly by the PT extract (t_{1/2}=10 [symbol] 2 min, n=3) than the RNA without the PTH mRNA insert (t_{1/2}=35 [symbol] 5 min, n=3) (FIG. 6). Insertion of the shorter PTH. RNA 38 nt, did not destabilize the chimeric transcript (t_{1/2}=40 [symbol] 5 min, n=3). These results suggest that the PTH 63 nt RNA destabilized the random RONA sequence of pCRII. This effect was similar to the effect of the 63 nt when it was inserted into a larger transcript, GH RNA, representing a cellular mRNA. The destablizing effect was dependent on an intact protein-binding transcript, because a shorter transcript that disrupted protein binding did not have the same effect.

### Example 5

### Purification of the PTH mRNA 3′-UTR Binding Proteins

To identify the proteins which bind to the PTH mRNA 3′-UTR, the inventors performed affinity chromatography. The proteins which bind the PTH mRNA 3′-UTR are present in all tissues examined [Moallem, E., ibid., (1998)]. Therefore, rat brain protein extracts and not the minute parathyroids, were used as the source for the RNA binding proteins. Rat brain S-100 extracts were chromatographed first on a heparin-sepharose column to enrich for proteins that bind RNA. The fractions which showed maximum binding to the PTH 3′-UTR on UV cross-linking were then chromatographed on a PTH RNA affinity column. The affinity column consisted of cyanogenbromide-activated sepharose linked to in vitro transcribed PTH RNA 3′-UTR. The proteins that bound the 3′-UTR column were eluted with increasing salt concentrations and studied by U.V. cross-linking to the PTH 3′-UTR RNA probe (FIG. 7A). There were three protein-RNA bands, at about 50, 60 and 110 kDa, for brain and parathyroid, consistent with our earlier studies [Moallem, E., ibid., (1998)]. The proteins that eluted between 220-500 mM NaCl exhibited maximum binding (FIG. 7A) and were combined and concentrated. A sample was run on an SDS-polyacrylamide gel and transferred to a nitrocellulose membrane, which was stained for protein by Ponceau. The staining revealed several bands (FIG. 7B). To identify the RNA-binding proteins, the membrane was then incubated with a riboprobe for the PTH 3′-UTR for Northwestern analysis (FIG. 7B). The PTH 3′-UTR showed the most intense binding to three of the proteins. There was one protein at approximately 60 kDA, two at about 50 kDa and other less intense bands.

PIT mRNA 3′-UTR Binding Proteins Stabilize the PTH RNA Transcript in an In Vitro Degradation Assay with Parathyroid Proteins

To demonstrate the function of the PTH mRNA 3′-UTR binding proteins on PTH mRNA stability in vitro degradation assays were performed. In the presence of cytosol there is gradual degradation of the transcript as seen previously. The effect of added eluate from the RNA column on the ability of PT protein extracts from hypophosphatemic rats to degrade PTH RNA in vitro was measured. Hypophosphatemic PT proteins showed more rapid degradation of PTH RNA in an in vitro degradation assay compared to PT proteins of control rats and also less binding to the PTH mRNA 3′UTR [Moallem, E., ibid., (1998)]. Proteins from hypophosphatemic PTs are therefore depleted in stabilizing factors. Complete depletion of these factors from PT cytosolic proteins by 3′-UTR affinity chromatography is not practicable because of the small size of the rat PT gland, which would require the use of >150 rats for each experiment. The degradation assay was therefore performed with PT proteins from hypophosphatemic rats and increasing amounts of eluate (˜200 and 400 ng of protein) from the RNA column. The added eluate had no effect upon transcript stability at lower concentrations, however, higher concentrations of added eluate stabilized the PTH transcript throughout the experiment (t_{1/2} 80 min: 30 min) FIG. 8). The same stabilizing effect was also found when the eluate was added to PT cytosolic extracts from control rats (not shown). These results show that proteins eluted from the RNA column stabilize the PTH transcript in vitro and that the eluate can overcome the degrading effect of the PT proteins from low phosphate rats, whose PT proteins show decreased binding to the PTH 3′ UTR.

One of the PTH mRNA 3′UTR Binding Proteins is AUF1

The eluate from the RNA column contained several proteins. One of the proteins at 50 kDa was present in the highest concentration (FIG. 7B). For this reason the 50 kDa protein was gel purified and microsequenced generating 5 peptide sequences of 10-17 residues each Data base search identified the polypeptide as being identical to AU-rich binding protein (AUF1) which is known to be important to the half-life of other mRNAs [reviewed in Wilson, G. M. and Brewer, G., Prog Nucleic Acid Res Mol Biol 62:257-291 (1999)]. The peptide sequences did not identify which of the AUF1 isoforms had been isolated. However, the binding assays suggests that the PTH RNA 3′-UTR bound all isoforms. One of these isoforms, p40^{AUF1}, was further studied.

The binding of recombinant AUF1 to the PTH RNA 3′-UTR was demonstrated by RNA electrophoretic mobility shift assay (REMSA). Recombinant P40^{AUF1} bound the PTH 3′-UTR labeled transcript resulting in a shift of the RNA probe (FIG. 9). The binding was enhanced by increasing concentrations of recombinant p40^{AUF1} (FIG. 9). Without protein the labeled transcript ran as two bands. These two bands may represent secondary structures of the RNA molecules because denaturing the RNA by heating it to 80° C. and then allowing it to re-nature at room temperature resulted in a single band on a polyacrylamide gel. This re-natured probe bound p40^{AUF1} the same as the untreated transcript (not shown). This indicates that p40^{AUF1} alone can bind the PTH 3′-UTR in the absence of other cytosolic proteins.

AUF1 Stabilizes the PTH RNA Transcript in an In Vitro Degradation Assay with Parathyroid Proteins

To demonstrate the function of AUF1 in PTH mRNA stability in vitro degradation assays were performed. The degradation assay was performed with PT proteins from hypophosphatemic rats and increasing amounts of p40^{AUF1}. When recombinant p40^{AUF1} was added to the degradation assay in the presence of hypophosphatemic PT proteins, there was stabilization of the PTH transcript, which was dependent upon the amount of recombinant p40^{AUF1} added (FIG. 10A). At 50 ng added AUF1 had no effect upon transcript stability, however, at higher concentrations addition of AUF1 stabilized the PTH transcript throughout the experiment (t_{1/2} of 90 min with AUF1:30 min without AUF1) (FIG. 10A). FIG. 10B shows the degradation of the PTH transcript with PT proteins from both normal and hypophosphatemic rats, where there is more rapid degradation with hypophosphatemic parathyroid proteins (t_{1/2} 30 nmi with hypophosphatemic PT proteins: 60 min with normal PT proteins). Addition of p40^{AUF1} to the hypophosphatemic proteins stabilized the transcript (t_{1/2}>120 In) even more than when the degradation assay was performed in the presence of proteins from normal rats. Control proteins had no effect (FIG. 10B). The control proteins used were bovine serum albumin (BSA) and dynein light chain (LC8). LC8 also binds to the PTH mRNA 3′-UTR [Epstein, E., et al., J Bone Miner Res 12:S132 (1997)].

To understand the effect of AUF1 and the other proteins in the eluate on the degradation reaction, recombinant p40^{AUF1} and the eluate were added to the reaction with hypophosphatemic proteins in concentrations where alone they had no effect on PTH RNA degradation (FIGS. 8 and 10A). The PTH RNA was now markedly stabilized (FIG. 10A). Therefore, there is an additive effect of p40^{AUF1} and the RNA binding proteins eluted from the RNA column.

### Example 6

### Screening of a Combinatorial Library for Potential Drugs

The 26 nucleotides cis element, the 40 nucleotide oligonucleotide and the 63 nucleotide comprising the cis element with its flanking sequences are examples of oligonucleotides capable of binding PT proteins to 3′-UTR of PTH mRNA. These oligonucleotides might serve as tools for high throughput screening of combinatorial libraries for substances capable of modulating PTH mRNA stability, leading to balanced levels of PTH mRNA and serum PTH. Screening for said substances will be done by standard methods such as screening of a combinatorial library on solid phase [Blaney and Martin, Curr Opin Chem Biol 1(1):54-59 (1997)].

### Example 7

### Tests for the Activity of Potential Drugs

After a target substance is isolated, it needs to be tested for its activity in vivo. This will be performed using a number of different assays:

1. In Vitro

Parathyroid cytosolic proteins will be used in the in vitro degradation assay radiolabeled PTH mRNA, as described in the preliminary results in Example 3. PT-proteins lead to a degradation of the PTH mRNA transcript. PT-proteins from rats fed a low calcium diet lead to a more stable transcript in this assay and PT-proteins from rats fed a low phosphate diet lead to a rapid degradation of the transcript [Moallem et al., ibid.]. To this assay will be added the test compound or analogues predicted to have no activity. Test compounds will be added to the assay with PT-proteins from rats fed both low phosphate diet and low calcium diets to test for increased stability, with the low phosphate diet PT-proteins, and decreased stability with the low calcium PT-proteins.

2. In Vivo

Rats or mice will be administered the test compounds in a single as well in divided doses, say 3×/day for 1 to 30 days and measurements then made. The parameters to be measured as serum concentration are: calcium, phosphate, alkaline phosphatase, osteocalcin, creatinine, urea, chloride and PTH. In addition, PTH mRNA levels will be measured. Once an effective dose is determined, then studies will also be performed on rats with experimental secondary hyperparathyroidism due to calcium deficiency or experimental uremia due to 5/6 nephrectomy. Further studies may be performed on mice after ovariectomy for the above parameters as well as bone density and histomorphometry.

### Example 8

### Clinical Uses of Potential Drugs

An artificial substance that mimics the function of PT proteins or, alternatively, increases the affinity of PTH mRNA to natural PT proteins might inhibit degradation of said mRNA, leading to sustained higher levels of PTH mRNA and serum PTH. Such effect is desired when treating patients who suffer loss of bone calcium due to low levels of serum calcium and loss of the circadian rhythm of PTH seen in patients with osteoporosis who do not have the normal increase in nocturnal PTH and phosphorus [Prank et al., J Clin Invest 95:2910-2919 (1995); Fraser et al., Clin Endocrinol (Oxf) 40:523-528 (1994); Portale et al., J Clin Invest 80:1147-1154 (1987)].

Another group of patients might benefit from PTH mRNA degradation. These people suffer chronic renal failure (CRF). Patients with CRF have increased activity of the PT gland with increased production of PTH. This leads to disabling bone disease as well as severe vascular disease. A major factor causing the increased PTH is the increased level of serum phosphate these patients have because they cannot excrete phosphate by their sick kidneys. The increased phosphate level and the accompanying decreases in serum 1α,25-dihydroxyvitamin D and calcium lead to an increase in PTH mRNA, serum PTH, and parathyroid cell proliferation. The phosphate and calcium both act post-transcriptionally on the 3′-UTR of PTH mRNA Drugs interacting with the element would prevent the increased PTH mRNA levels and serum PTH, by preventing interaction of PT proteins with 3′-UTR of PTH mRNA.

1. An isolated cis-acting regulatory nucleic acid sequence comprising a 63 nucleotide fragment of the 3′-UTR of parathyroid hormone (PTH) gene and allelic variations, mutations or functionally equivalent fragments thereof, wherein said sequence, when operably linked to a heterologous or homologous coding sequence of interest, is capable of directing specific regulation of stability of the mRNA encoded by said heterologous or homologous coding sequence of interest.

2. The isolated cis-acting sequence according to claim 1, wherein regulation of the stability of said mRNA is responsive to changes in serum levels of any one of calcium and phosphate.

3. The isolated cis-acting sequence according to claim 2, wherein the regulation of the mRNA stability is further mediated by the binding of at least one PT protein or derivatives thereof to said cis-acting sequence.

4. The isolated cis-acting sequence according to claim 3, wherein said 63 nucleotide functional fragment consists of consecutive nucleotides having the sequence substantially as denoted by any one of SEQ ID NO:1 and NO:8 and allelic variations, mutations or functionally equivalent fragments thereof.

5. The isolated cis-acting sequence according to claim 4, wherein said functional fragment is a 40-nucleotide sequence substantially as denoted by any one of SEQ ID NO:2 and NO:9 and allelic variations, mutations or functionally equivalent fragments thereof.

6. The isolated cis-acting sequence according to claim 5, wherein said functional fragment is a 26-nucleotide sequence substantially as denoted by any one of SEQ ID NOs:3 to 7 and 10 to 14 and allelic variations, mutations or functionally equivalent fragments thereof.

7. The isolated cis-acting sequence according to any one of claims 1 to 6, wherein said nucleic acid sequence is further operably linked to heterologous or homologous coding sequence and optionally to additional control, promoting and/or regulatory elements.

8. The isolated cis-acting sequence according to claim 7, wherein said heterologous or homologous coding sequence encodes a protein selected from the group consisting of reporter proteins, enzymes, hormones, growth factors, cytokines, structural proteins and industrially applicable proteins, or is itself a therapeutic product.

9. The isolated cis-acting sequence according to claim 8, wherein said homologous coding sequence is the parathyroid hormone (PTH) coding sequence, substantially as denoted by the GenBank Accession No. X05721.

10. The isolated cis-acting sequence according to claim 8, wherein said heterologous coding sequence encodes a reporter protein selected from the group consisting of green fluorescent protein (GFP), luciferase, secreted alkaline phosphatase (SEAP), β-galactosidase (β-gal), β-glucoronidase and a secreted protein.

11. The isolated cis-acting sequence according to claim 10, wherein said secreted protein is GH (growth hormone).

12. A DNA construct comprising:
a. an isolated cis-actin regulatory nucleic acid sequence comprising a 63 nucleotide fragment of the 3′-UTR of parathyroid hormone (PTH) gene and allelic variations, mutations or functionally equivalent fragments thereof, wherein said sequence, when operably linked to a heterologous or homologous coding sequence of interest, is capable or directing specific regulation of stability of the mRNA encoded by said heterologous or homologous coding sequence of interest;
b. a heterologous or homologous coding sequence operably linked to said isolated cis-acting sequence of (a); and optionally
c. additional control, promoting and/or regulatory elements.

13. The DNA construct according to claim 12, wherein said 63 nucleotide functional fragment consists of consecutive nucleotides having the nucleotide sequence substantially as denoted by SEQ ID NO:8 and allelic variations, mutations or functionally equivalent fragments thereof.

14. The DNA construct according to claim 13, wherein said functional fragment is a 40-nucleotide sequence substantially as denoted by SEQ ID NO: 9 and allelic variations, mutations or functionally equivalent fragments thereof.

15. The DNA construct according to claim 14, wherein said functional fragment is a 26-nucleotide sequence substantially as denoted by any one of SEQ ID NOs:10 to 14 and allelic variations, mutations or functionally equivalent fragments thereof.

16. The DNA construct according to any one of claims 12 to 15, wherein said heterologous or homologous coding sequence encodes a protein selected from the group consisting of reporter proteins, enzymes, hormones, growth factors, cytokines, structural proteins and industrially applicable proteins, or said protein is itself a therapeutic product.

17. The DNA construct according to claim 16, wherein said homologous coding sequence is the parathyroid hormone (PTH) coding sequence, substantially as denoted by GenBank Accession No. X05721.

18. The DNA construct according to claim 16, wherein said heterologous coding sequence encodes a reporter protein selected from the group consisting of green fluorescent protein (GFP), luciferase, secreted alkaline phosphatase (SEAP), β-galactosidase (β-gal), β-glucoronidase and a secreted protein.

19. The DNA construct according to claim 18, wherein said secreted protein is GH (growth hormone).

20. An expression vector comprising a cis-acting regulatory nucleic acid sequence according to any one of claims 1 to 11 or a DNA construct according to any one of claims 12 to 19 and a suitable DNA carrier, capable of transfecting a host cell with said cis-acting regulatory nucleic acid sequence.

21. An expression vector according to claim 20, further comprising additional expression, control, promoting and/or regulatory elements operably linked thereto.

22. A host cell transfected with a DNA construct according to any one of claims 12 to 19.

23. AR host cell transfected with an expression vector according to any one of claims 20 and 21.

24. A host cell according to any one of claims 22 and 23, being a eukaryotic cell.

25. A host cell according to claim 24, wherein said eukaryotic cell is a mammalian cell.

26. A host cell according to claim 25, wherein said mammalian cell is a PT cell.

27. A host cell according to claim 25, wherein said mammalian cell is selected from the group consisting of COS7, HEK (293T) and CHO cell lines.

28. A complex comprising a cis-acting regulatory nucleic acid sequence according to claim 1 bound to at least one PT protein.

29. A complex comprising a cis-acting regulatory nucleic acid sequence according to claim 1 bound to at least one PT-protein-mimetic agent.

30. A complex comprising a cis-acting regulatory nucleic acid sequence according to any one of claims 4 to 6 bound to at least one PT protein.

31. A complex comprising a cis-acting regulatory nucleic acid sequence according to any one of claims 4 to 6 bound to at least one PT-protein-mimetic agent.

32. An agent that selectively binds the cis-acting regulatory nucleic acid sequence according to any one of claims 4 to 6.

33. An agent according to claim 32, capable of enhancing the affinity of a cis-acting regulatory nucleic acid sequence according to any one of claims 4 to 6 to at least one PT protein.

34. An agent capable of modulating the affinity of a cis-acting regulatory nucleic acid sequence according to any one of claims 4 to 6 to at least one PT protein.

35. An agent according to claim 34, capable of enhancing the affinity of a cis-acting regulatory nucleic acid sequence according to any one of claims 4 to 6 to at least one PT protein.

36. An agent according to claim 34, capable of decreasing the affinity of a cis-acting regulatory nucleic acid sequence according to any one of claims 4 to 6 to at least one PT protein.

37. An agent capable of affecting the stability of a complex according to any one of claims 28 to 31.

38. A method of screening for substances that specifically bind to a cis-acting regulatory nucleic acid sequence comprising any of the functional fragments of the 3′ untranslated region (UTR) of the gene encoding parathyroid hormone (PTH) as defined in any one of claims 1 to 6, comprising the steps of:
(a) providing a sample containing a combinatorial library of candidate substances,
(b) depositing said cis-acting regulatory nucleic acid sequence on a suitable solid phase carrier;
(c) incubating the said sample with the deposited cis-acting regulatory nucleic acid sequence obtained in step (b);
(d) washing off any non-bound sample material;
(e) separating bound material from said solid phase carrier; and
(f) identifying the material obtained in step (e).

39. A method according to claim 38 for screening for agents which affect the stability of a complex as defined in any one of claims 28 to 31.

40. A method of screening for substances that specifically bind to an isolated cis-acting regulatory nucleic acid sequence comprising any of the functional fragments of the 3′UTR of parathyroid hormone (PTH) gene and allelic variations, mutations or functionally equivalent fragments thereof as defined in any one of claims 1 to 6, wherein said binding affects the regulation of mRNA stability by said cis-acting sequence, which method comprises the steps of:
a. providing a host cell transformed with any one of an expression vector and a DNA construct comprising:
(i) said isolated cis-acting sequence;
(ii) a heterologous or homologous coding sequence operably linked to said sequence in (i); and
(iii) operably linked additional control, promoting and/or regulatory elements;
b. introducing a combinatorial library of candidate substances to said host cell, under conditions which lead to the regulation of stability of the mRNA encoded by said heterologous or homologous coding sequence of interest;
c. deflecting an end point indicative of regulation of stability of said mRNA, wherein said regulation is affected by binding of said candidate substances expressed by a certain clone of the combinatorial library, to said cis-acting sequence; and
d. isolating said combinatorial library clones expressing a substance that binds said cis-acting sequence and affects the regulation of mRNA stability by said isolated cis-acting sequence.

41. A method of screening for a substance which affects regulation of mRNA stability by an isolated cis-acting regulatory nucleic acid sequence comprising any of the functional fragments of the 3′-UTR of parathyroid hormone (PTH) gene and allelic variations, mutations or functionally equivalent fragments thereof as defined in any one of claims 1 to 6, comprising the steps of:
a. providing a host cell transformed with any one of an expression vector and a DNA construct comprising:
(i) said isolated cis-acting sequence; and
(ii) heterologous or homologous coding sequence operably linked to said sequence in (i); and
(iii) operably linked additional control, promote, and/or regulatory elements;
b. exposing said host cell to a test substance under conditions which lead to the regulation of stability of the mRNA encoded by said heterologous or homologous coding sequence of interest; and
c. detecting an end point indicative of regulation of stability of said mRNA, affected by said test substance.

42. The method of any one of claims 40 and 41, wherein said cis-acting sequence, when operably linked to a heterologous or homologous coding sequence of interest, is capable of directing specific regulation of stability of the mRNA encoded by said heterologous or homologous coding sequence of interest, according to any of claims 1 to 11.

43. The method of claim 42, wherein said host cell is according to any one of claims 22 to 27.

44. The method of any one of claims 40 to 43 wherein said indicative end point is the expression of said operably linked heterologous or homologous coding sequence.

45. The method of claim 44, wherein said homologous coding sequence is the parathyroid hormone (PTH) coding sequence, substantially as denoted by the GenBank Accession No. X06721.

46. The method of claim 44, wherein said heterologous coding sequence encodes a reporter protein selected from the group consisting of green fluorescent protein (GFP), luciferase, secreted alkaline phosphatase (SEAP), β-galactosidase (β-gal), β-glucoronidase and a secreted protein.

47. The method according to claim 46, wherein said secreted protein is GH (growth hormone).

48. The method of claim 47, wherein expression of the gene encoding said reporter protein leads to a visually detectable signal.

49. A pharmaceutical composition for the prevention and/or treatment of a disorder associated with abnormal function of parathyroid gland, comprising as active ingredient a therapeutically effective amount of at Least one natural PT protein.

50. A pharmaceutical composition for the prevention and/or treatment of a disorder associated with abnormal function of parathyroid, comprising as active ingredient a therapeutically effective amount of at least one agent according to claims 32 to 37.

51. A pharmaceutical composition for the prevention and/or treatment of a disorder associated with abnormal function of parathyroid gland, comprising as an active ingredient a therapeutically effective amount of at least one agent according to any one of claims 32 to 37.

52. A pharmaceutical composition according to claims 49 to 51, for the treatment and/or prevention of overproduction or underproduction of PTH.

53. A pharmaceutical composition for the treatment of a disorder associated with abnormal metabolism of or calcium and/or phosphate, comprising as an active ingredient a therapeutically effective amount of at least one agent according to claims 32 to 37.

54. A pharmaceutical composition for the treatment of a disorder associated with abnormal metabolism of or calcium or phosphate, comprising as an active ingredient a therapeutically effective amount of at least one agent according to any one of claims 32 to 37.

55. A pharmaceutical composition according to claims 48 to 51, for the treatment and/or prevention of bone diseases, particularly osteoporosis.

56. A pharmaceutical composition according to claims 48 to 51, for the treatment of chronic renal failure (CRF).

57. An antiserum containing antibodies directed against an agent according to claims 32 to 37.


SEQUENCE LISTING (25 sequences)

SEQ ID NO: 1 - RNA, 63 nt, RATTUS NORVEGICUS
gucucuuccaaugauuccauuucaauauauucuucuuuuuaaaguauuacacauuuccacuuc

SEQ ID NO: 2 - RNA, 40 nt, RATTUS NORVEGICUS
aaugauuccauuucaauauauucuucuuuuuaaaguauua

SEQ ID NO: 3 - RNA, 26 nt, RATTUS NORVEGICUSI
aauauauucuucuuuuuaaaguauua

SEQ ID NO: 4 - RNA, 26 nt, HOMO SAPIENS
uauuguuuauucuuuuuaaaguaugu

SEQ ID NO: 5 - RNA, 26 nt, MUS MUSCULUS
aauaugcucuucuuuuuaaaguacua

SEQ ID NO: 6 - RNA, 26 nt, CANIS DOMESTICUS
uauuguuuauucuuuuuaaaguaugu

SEQ ID NO: 7 - RNA, 26 nt, SUS SCROFA
aauauauucuucuuuuuaaaguauua

SEQ ID NO: 8 - DNA, 63 bp, RATTUS NORVEGICUS
gtctcttccaatgattccatttcaatatattcttctttttaaagtattacacatttccacttc

SEQ ID NO: 9 - DNA, 40 bp, RATTUS NORVEGICUS
aatgattccatttcaatatattcttctttttaaagtatta

SEQ ID NO: 10 - DNA, 26 bp, RATTUS NORVEGICUS
aatatattcttctttttaaagtatta

SEQ ID NO: 11 - DNA, 26 bp, HOMO SAPIENS
tattgtttattctttttaaagtatgt

SEQ ID NO: 12 - DNA, 26 bp, MUS MUSCULUS
aatatgctcttctttttaaagtacta

SEQ ID NO: 13 - DNA, 26 bp, CANIS DOMESTICUS
tattgtttattctttttaaagtatgt

SEQ ID NO: 14 - DNA, 26 bp, SUS SCAROFA
aatatattcttctttttaaagtatta

SEQ ID NO: 15 - RNA, 99 nt, RATTUS NORVEGICUS
gucucuuccaaugauuccauuucaauauauucuucuuuuuaaaguauuacacauuuccacuucucuccuuaaauauaaauaaaguuuaaugaucaugaa

SEQ ID NO: 16 - DNA, 15 bp, Artificial Sequence
  Description of Artificial Sequence5′ PRIMER FOR 100 TRANSCRIPT
gtctcttccaatgat

SEQ ID NO: 17 - DNA, 20 bp, Artificial Sequence
  Description of Artificial Sequence3′ PRIMER FOR 100 TRANSCRIPT
ttcatgatcattaaacttta

SEQ ID NO: 18 - DNA, 24 bp, Artificial Sequence
  Description of Artificial Sequence3′ PRIMER FOR 63 TRANSCRIPT
aagtggaaatgtgtaatactttaa

SEQ ID NO: 19 - RNA, 50 nt, RATTUS NORVEGICUS
gucucuuccaaugauuccauuucaauauauucuucuuuuuaaaguauuac

SEQ ID NO: 20 - DNA, 27 bp, Artificial Sequence
  Description of Artificial Sequence3′ PRIMER FOR 50 TRASCRIPT
taatactttaaaaagaagaatatattg

SEQ ID NO: 21 - DNA, 19 bp, Artificial Sequence
  Description of Artificial Sequence5′ PRIMER FOR THE 40 TRANSCRIPT
aatgattccatttcaatat

SEQ ID NO: 22 - DNA, 18 bp, Artificial Sequence
  Description of Artificial Sequence3′ PRIMER FOR 40 TRANSCRIPT
taatactttaaaaagaag

SEQ ID NO: 23 - DNA, 51 bp, Artificial Sequence
  Description of Artificial Sequence5′  PRIMER FOR 30 TRANSCRIPT
attaaccctcactaaagggacatttcaatatattcttctttttaaagtatt

SEQ ID NO: 24 - DNA, 31 bp, Artificial Sequence
  Description of Artificial Sequence3′ PRIMER FOR THE 30 TRANSCRIPT
aatactttaaaaagaagaatatattgaaatg

SEQ ID NO: 25 - DNA, 47 bp, Artificial Sequence
  Description of Artificial Sequence5′ PRIMER FOR THE 26 TRANSCRIPT
attaaccctcactaaagggacaatatattcttctttttaaagtatta